(19) United States
(12) Patent Application Publication
(10) Pub. No.: US 2016/0058286 A1
(43) Pub. Date: Mar. 3, 2016

(54) DISTRIBUTED HEALTH CARE MONITORING

(71) Applicant: Covidien LP, Mansfield, CO (US)

(72) Inventors: DEV JOSHUA, SUPERIOR, CO (US); SCOTT HUNSAKER, Boulder, CO (US); JAN STEPHEN BELCHER, Arvada, CO (US); THOMAS WILMERING, Westminster, CO (US)

(21) Appl. No.: 14/840,375

(22) Filed: Aug. 31, 2015

Related U.S. Application Data
(60) Provisional application No. 62/044,815, filed on Sep. 2, 2014.

(51) Int. Cl. A61B 5/00

(52) U.S. Cl. CPC A61B 5/0022; A61B 5/742; A61B 5/7475

(57) ABSTRACT

Methods, apparatuses and systems are described for distributed health care monitoring. In one example method, a plurality of functions associated with a health care monitoring process are defined and distributed among a plurality of health care devices. The plurality of health care devices communicate using a network connecting the health care devices to monitor a patient.

### CROSS REFERENCES

The present disclosure claims priority to U.S. Provisional Patent Application No. 62/044,815 entitled “Distributed Health Care Monitoring” and filed on Sep. 2, 2014, the entirety of which is incorporated herein by reference.

### TECHNICAL FIELD

The present disclosure relates generally to health care monitoring, and more particularly to systems and methods for distributed health care monitoring.

### BACKGROUND

Conventional health care monitoring systems typically include tightly coupled hardware and software components that are frequently only able to function with other specific components from the same manufacturer. For example, a bedside monitor for measuring oxygen levels in a patient may include a sensor, data processing equipment, and a display, all made by a single manufacturer and connected using proprietary connectors. The data measured by the sensor is received and analyzed by the data processing equipment, and then shown on the display so that it can be interpreted by a health care provider. The measured data usually cannot, however, be provided to other devices, and the display of the monitor is typically unable to display data measured by other sensors of other health care monitoring devices.

This tight coupling of the various components can reduce the ability to re-use certain components for several different monitoring functions, such as the ability to use one display to provide graphical information from many different sensors or other monitoring devices, or the ability to provide measurement data from one sensor to many different processers for use and analysis. The proprietary nature of the components can also reduce portability—for example, a user may need to purchase many different components from a single manufacturer because equipment from one manufacturer may not be interoperable with equipment from a second manufacturer. Moreover, the development, manufacture, and maintenance of these tightly coupled, proprietary health care monitoring devices may be difficult due to the complexity of each device and system.

### SUMMARY

The described features generally relate to one or more improved methods, systems, and devices for distributed health care monitoring. The functions of a comprehensive health care monitoring process may divided into individual, functional and/or logical modules, which may be implemented in software and/or hardware and distributed throughout one or more different health care devices. In some embodiments, no single health care device implements all of the monitoring functions, but instead two or more separate health care devices work together to provide the collective functionality needed in a particular health care monitoring situation. Also, in some embodiments, the health care devices may be remotely managed in order to, for example, determine usage charges, business intelligence information, and so forth.

A method for health care monitoring is thus described, with the method including defining a number of functions associated with a health care monitoring process, distributing the functions associated with the health care monitoring process among a number of health care devices, and communicating among the health care devices using a network connecting the health care devices to monitor a patient.

In some examples, distributing the functions among the health care devices may involve including one or more monitoring modules implementing respective ones of the defined functions in each of the health care devices. Further, the method may further include defining a number of different topics of data that can be published by each of the respective monitoring modules, wherein the different topics are defined according to a data distribution service protocol. The health care devices may cooperatively work together to accomplish all of the functions associated with the health care monitoring process. In some examples the network may be a wireless network between at least two of the health care devices. The network may define a data bus to which each of the health care devices may publish certain types data and from which each of the health care devices may subscribe to certain types of data. Also, each health care device that subscribes to a certain type of data may be agnostic as to which other health care device published that certain type of data.

In some examples, each of the health care devices may be interoperable with a number of types of different health care devices. Also, the health care devices may include first and second health care devices, the first and second health care devices may each include a first monitoring function, and the method may further include utilizing the first health care device to implement the first function during a first time period, and switching to utilize the second health care device to implement the first function during a second time period upon an occurrence of a pre-defined event. The second health care device may not be utilized during the first time period to implement the first function and the first health care device may not be utilized during the second time period to implement the first function. In some examples, the pre-defined event may relate to loss of power for the first health care device. Also, the health care devices may further include a third health care device including a second monitoring function, and the third health care device may be utilized to implement the second monitoring function during both the first and second time periods. The switching to utilize the second health care device may happen automatically upon the occurrence of the pre-defined event.

Also disclosed is a health care device, which includes a monitoring module configured to perform one of a number of defined functions associated with a health care monitoring process and a core domain module, wherein the health care device is configured to communicate with other health care devices over a network, the other health care devices including modules configured to perform others of the defined functions associated with the health care monitoring process.

In some examples, the monitoring module may be a data source domain module configured to transmit measurements from a transducer to the communications module for publication on the network. The health care device may further include a communications interface configured to communicate with the other health care devices over the network. The monitoring module may be a user interface module configured to display information received over the network via the communications module.

A method for health care monitoring is also disclosed, the method including incorporating at least one function of a number of defined functions associated with a health care monitoring process into at least one health care device of a number of health care devices, and utilizing the at least one health care device to perform a subset of the defined functions associated with the health care monitoring process.

A distributed health care monitoring system is also disclosed, the system including a number of health care devices, each of the health care devices including two or more of a number of monitoring modules, each of the monitoring modules configured to implement a respective function associated with a health care monitoring process, and a network connecting the health care devices.

In some examples, the network may define a data bus to which each of the monitoring modules may publish certain types data and from which each of the monitoring modules may subscribe to certain types data. The system may also include a server coupled with the network and configured to store information published to the data bus by the health care devices. One of the health care devices may include a sensor device, the sensor device may include a data source domain module and a transducer, and the data source domain module may be configured to publish measurements from the transducer onto the data bus. One of the health care devices may include a display device, the display device may include a user interface domain module and a display, and the user interface domain module may be configured to subscribe to the measurements from the data source domain module of the sensor device and further configured to display the measurements on the display. In some examples, none of the health care devices includes all of the monitoring modules. Also, the monitoring modules may include a core domain module, a remote device management domain module, a data source domain module, a parameter domain module, an alarm domain module, a trend domain module, a user interface domain module, a hardware interfaces domain module, and an algorithm module.

A multi-platform health care monitoring system is also disclosed, the system including a source code database including a number of generic software modules, each of the generic software modules configured to implement a respective function associated with a health care monitoring process, and a compiler module configured to compile each of the generic software modules into executable code modules corresponding to a number of different operating systems.

In some examples, the different operating systems may be associated with a respective number of different health care monitoring devices. At least two of the different health care monitoring devices may be from different manufacturers. Also, each of the executable code modules may be configured to publish data to a common data bus in a common data format regardless of the operating system corresponding to the respective executable code module. Each executable code module may include components common to the health care monitoring process, components common to a monitoring domain associated with the executable code module, and implementation specific components for the executable code module itself.

A method for managing a health care device is also disclosed, the method including selectively coupling a health care device with a network for remote device management of the health care device, and receiving instructions from or sending information to a remote device management server relating to the health care device.

In some examples, the network may be one or more of a wireless local area network, a wireless wide area network, a wired local area network, or the Internet. The remote device management server may be located remotely from the health care device, and the selective coupling may include supplying a network connection between the health care device and the remote device management server. In some examples, the method may further include utilizing a remote device management tool as an intermediary between the health care device and the remote device management server. The method may also include establishing a connection between the remote device management tool and the health care device and/or reconciling the remote device management tool with the remote device management server. Information may be sent to the remote device management server, and the information may include usage information for the health care device. In some examples, billing information may be generated based on the usage information.

In some examples, instructions may be received from the remote device management server, and the instructions may include one or more of licensing information, updating information, or testing information. The selective coupling of the health care device with the network may be automatically performed if the health care device is detected on a wireless network. Also, in some examples, the method may further include automatically updating the health care device once the health care device is selectively coupled with the wireless network.

In some examples, instructions may be received from the remote device management server and the instructions may include an activation instruction for demoing an unlicensed function that the health care device is capable of performing. The method may also include determining relative costs associated with a number of different network technologies for receiving the instructions or sending the information, and utilizing the determined relative costs to select one of the different network technologies for receiving the instructions from or sending the information to the remote device management server. At least one of the health care devices may include a database configured to store data, and the method may further include transmitting stored data from the database to the remote device management server.

In some examples, at least one of the health care devices may not include a database configured to store data, and the method may further include transmitting data generated by the at least one of the health care devices to the remote device management server in real-time. Also, an instruction may be received at the health care device from the remote device management server to transmit a usage log associated with the health care device, in which case the method may further include transmitting the usage log to the remote device management server.

A method for managing a health care device is also disclosed, the method including sending instructions from a remote device management server to a health care device coupled with the remote device management server via a network, and receiving information related to the health care device at the remote device management server based on the sent instructions.

A method for charging for use of a health care device is also disclosed, the method including receiving information regarding one or more identifiable uses of a health care device, determining a charge corresponding to each identifiable use of the health care device, and generating a billing report based on the determined charge for each identifiable use of the health care device.

In some examples, the received information may include a location within a health care facility in which the health care device was used, and the charge corresponding to the identifiable use may be based on the location. Different locations within the health care facility may be associated with different charges for use of the health care device. In some examples, the received information may include a parameter monitored by the health care device, and the charge corresponding to the identifiable use may be based on the parameter monitored by the health care device. The received information may alternatively or additionally include a time at which the health care device was used, and the charge corresponding to the identifiable use may be based on the time. In some examples, the received information may include an outcome of using the health care device, and the charge corresponding to the identifiable use may be based on the outcome. For example, the outcome may be an improvement in delivery of health care services. Also, in some examples, the health care device may be supplied at no charge.

A method for analyzing data from a health care device is also disclosed, the method including receiving data from a health care device, processing the received data at a location remote from the health care device to generate business intelligence based on the received data, and transmitting the business intelligence.

In some examples, the business intelligence may be transmitted to a user of the health care device and/or to a server for review by an administrator of the health care device. The received data may include usage information and the business intelligence may include trend information. The received data may alternatively or additionally include usage information and the business intelligence may include one or more suggested training opportunities. The received data may alternatively or additionally include one or more parameters monitored by the health care device, and the business intelligence may include decision support for a health care provider.

In some examples, the data may be continuously received from the health care device. Alternatively, the data may be buffered at the health care device and received at the remote location at predefined times. Also, in some examples, the data may be received from the health care device only after it is requested by the remote location. The data received from the health care device may include a summary of a data set available at the health care device. Also, in some examples, the data may be received over one or more of a wireless local area network connection, a wireless wide area network connection, a wired local area network connection, or the Internet.

Further scope of the applicability of the described methods and systems will become apparent from the following detailed description, claims, and drawings. The detailed description and specific examples are given by way of illustration only, since various changes and modifications within the spirit and scope of the description will become apparent to those skilled in the art.

### BRIEF DESCRIPTION OF THE DRAWINGS

A further understanding of the nature and advantages of the present invention may be realized by reference to the following drawings. In the appended figures, similar components or features may have the same reference label. Further, various components of the same type may be distinguished by following the reference label by a dash and a second label that distinguishes among the similar components. If only the first reference label is used in the specification, the description is applicable to any one of the similar components having the same first reference label irrespective of the second reference label.

FIG. 1 is a block diagram of a distributed health care monitoring system in accordance with aspects of the present disclosure;

FIG. 2A is a block diagram of a health care device with a number of monitoring modules in accordance with aspects of the present disclosure;

FIG. 2B is a block diagram of a health care device with a number of monitoring modules in accordance with aspects of the present disclosure;

FIG. 2C is a block diagram of a virtual network with a number of health care devices in accordance with aspects of the present disclosure;

FIG. 2D is a block diagram of a virtual network with a number of health care devices in accordance with aspects of the present disclosure;

FIG. 2E is a block diagram of multiple virtual networks in a health care facility in accordance with aspects of the present disclosure;

FIG. 3 is a block diagram of a health care device with a number of monitoring modules in accordance with aspects of the present disclosure;

FIG. 4 is a block diagram of a health care device with a number of monitoring modules in accordance with aspects of the present disclosure;

FIG. 5 is a block diagram of a distributed health care monitoring system in accordance with aspects of the present disclosure;

FIG. 6 is a block diagram of a distributed health care monitoring system in accordance with aspects of the present disclosure;

FIG. 7 is a block diagram of a distributed health care monitoring system in accordance with aspects of the present disclosure;

FIG. 8 is a block diagram of a server for use with a distributed health care monitoring system in accordance with aspects of the present disclosure;

FIG. 9 is a flow chart illustrating a method for health care monitoring in accordance with aspects of the present disclosure;

FIG. 10 is a flow chart illustrating a method for health care monitoring in accordance with aspects of the present disclosure;

FIG. 11 is a block diagram of a multi-platform health care monitoring system in accordance with aspects of the present disclosure;

FIG. 12 is a block diagram of an operating system specific executable code module in accordance with aspects of the present disclosure;

FIG. 13 is a block diagram of a health care device with a number of specific implementations of monitoring modules in accordance with aspects of the present disclosure;

FIG. 14 is a flow chart illustrating a method for compiling operating system specific monitoring modules in accordance with aspects of the present disclosure;

FIG. 15 is a block diagram of a system for managing one or more health care devices in accordance with aspects of the present disclosure;

FIG. 16 is a block diagram of a system for managing one or more health care devices in accordance with aspects of the present disclosure;

FIG. 17 is a flow chart for illustrating a method for managing one or more health care devices in accordance with aspects of the present disclosure;

FIG. 18 is a flow chart for illustrating a method for managing one or more health care devices in accordance with aspects of the present disclosure;

FIG. 19 is a block diagram of a system for charging for use of one or more health care devices in accordance with aspects of the present disclosure;

FIG. 20 is a block diagram of a system for charging for use of one or more health care devices in accordance with aspects of the present disclosure;

FIG. 21 is a flow chart illustrating a method for charging for use of one or more health care devices in accordance with aspects of the present disclosure;

FIG. 22 is a block diagram for analyzing data from one or more health care devices in accordance with aspects of the present disclosure;

FIG. 23 is a block diagram for analyzing data from one or more health care devices in accordance with aspects of the present disclosure; and

FIG. 24 is a flow chart illustrating a method for analyzing data from one or more health care devices in accordance with aspects of the present disclosure.

### DETAILED DESCRIPTION

Distributed health care monitoring systems, devices, and methods are described, wherein a number functions associated with a comprehensive health care monitoring process are defined and implemented in one or more health care devices. In some embodiments, the responsibility to carry out two or more of the defined functions is distributed among two or more health care devices that work together to provide the collective functionality needed in a particular health care monitoring context. A network coupling the two or more health care devices may be used so that the health care devices can share data and otherwise communicate.

In some embodiments, each defined function may be implemented in a monitoring module, with each health care device including one or more monitoring modules. The monitoring modules may include software code that is specific to the host health care device, and the software code may be generated by a multi-platform system that generates executable code for several different specific operating systems based on a common core defining each module's general functionality. Also, in some embodiments, the health care devices may be remotely managed by a remote device management server, optionally through an intermediary remote device management tool such as a tablet computer. A remote processing center may determine charges and billing reports for use of the health care devices, or, in other examples, may generate business intelligence (such as decision support) based on data received from the health care devices.

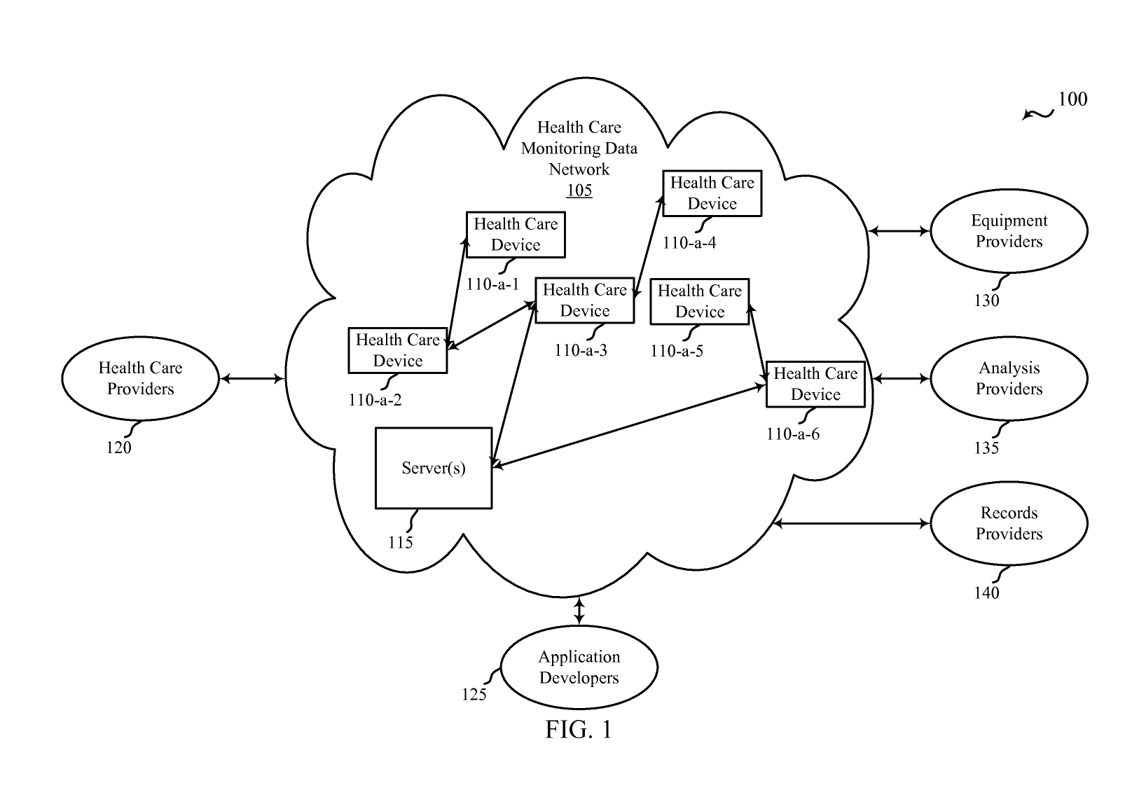

Referring first to FIG. 1, a diagram illustrates one example of a distributed health care monitoring system 100. The distributed health care monitoring system 100 includes a health care monitoring data network 105 with a number of health care devices 110 and optionally one or more servers 115. The health care devices 110 may include devices such as sensors, monitors, displays, tablet computers, smart phones, ventilators, staplers, infusion and feeding apparatuses, anesthesia or surgery apparatuses, bar code readers, and so forth. Generally, a health care device, as used herein, is any hardware and/or software that can be used to provide one or more health care monitoring functions (including sensing, parsing, interpreting, and displaying patient data, but also including accessories providing auxiliary services, functions related to patient treatment, and so forth), as described in more detail below.

Each of the health care devices 110 may perform one or more aspects of a health care monitoring process, and the health care monitoring data network 105 may allow the health care devices 110 to communicate and share data. The one or more servers 115 may be coupled with the network and configured to store information published by the health care devices 110 to a data bus (or data space) of the network 105. Alternatively, in some embodiments, the system 100 may not include any servers. In these embodiments, the “local only” system may provide for device-to-device or node-to-node connections between the health care devices 110. As also shown in FIG. 1, in some embodiments, health care providers 120, application developers 125, equipment providers 130, analysis providers 135, and/or records providers 140 may have access to the health care monitoring data network 105 in order to provide services associated with the health care monitoring system 100. In those embodiments without a server, the health care providers 120, application developers 125, equipment providers 130, analysis providers 135, and/or records providers 140 may have access to a shared data space among the health care devices 110 and/or direct access to the health care devices 110 themselves.

Each of the health care devices 110 and the servers 115 may be implemented with one or more application-specific integrated circuits (ASICs) adapted to perform some or all of the functions described herein in hardware. Alternatively, the functions may be performed by one or more other processing units (or cores), on one or more integrated circuits. In other embodiments, other types of integrated circuits may be used (e.g., Structured/Platform ASICs, Field Programmable Gate Arrays (FPGAs), and other Semi-Custom ICs), which may be programmed in any manner known in the art. The functions may also be implemented, in whole or in part, with instructions embodied in a memory (e.g., a software module), formatted to be executed by one or more general or application-specific processors.

The health care monitoring data network 105 in FIG. 1 may include one or more technologies for coupling the health care devices 110 and servers 115 (including both direct and indirect connections). The network 105 may include wired connections (e.g., Ethernet, twisted pair, etc.) and/or wireless connections (e.g., wireless wide area network (WWAN), wireless local area network (WLAN), Mesh network, Near Field Communication, Thread wireless protocol, ultra wide band, etc.), and may be implemented over a private network, a public network (e.g., the Internet), or some combination of private and public networks. As just one example, a first health care device 110-a-1 may be connected via a twisted pair wired connection to a second health care device 110-a-2, which in turn is connected to a third health care device 110-a-3 via a wireless Wi-Fi connection. A fourth health care device 110-a-4 may also be connected to the third health care device 110-a-3 via the wireless Wi-Fi connection. The third health care device 110-a-3 may be coupled with the server 115 via an Ethernet connection. Also, a fifth health care device 110-a-5 may be connected to a sixth health care device 110-a-6 via a short range, low power wireless connection, such as Bluetooth, and the sixth health care device 110-a-6 may be connected to the server 115 via the Internet. The providers 120, 130, 135, 140 may be able to access data from any of the health care devices 110 and/or the server 115 using an Internet or other connection to the network 105. It will be appreciated that many configurations of the health care monitoring data network 105 are contemplated beyond those explicitly illustrated in FIG. 1. For example, all of the health care devices 110 may be connected to the server 115 using individual Bluetooth connections, with the server 115 allowing health care providers 120 to access data from the health care devices, optionally via the server 115, using a private network operated by a health care facility. As described above, however, some systems 100 do not have any servers.

The health care monitoring data network 105 may define (e.g., include) a data bus to which each of the health care devices 110 may publish certain types of data and from which each of the health care devices 110 may subscribe to certain types of data. The data bus may be implemented as a shared physical medium (e.g., wires, an air interface, etc.) to which each of the health care devices 110 is coupled, may be implemented in a database stored in the server 115, or some combination thereof. For example, and referring again to the example shown in FIG. 1, the connection between the third health care device 110-a-3 and the second and fourth health care devices 110-a-2, 110-a-4 may define a shared Wi-Fi wireless data channel, to which each of the second, third, and fourth health care devices 110-a-2, 110-a-3, 110-a-4 can publish certain data. The shared data channel may implement a listen before talk procedure whereby the health care devices 110-a-2, 110-a-3, 110-a-4 listen to the shared data channel before publishing data to be sure that no other health care device is currently publishing data to the shared data channel. In other embodiments, however, the shared data channel may not implement a listen before talk procedure, and instead the health care devices 110-a-2, 110-a-3, 110-a-4 may publish data to the shared data channel regardless of whether the channel is open and available or not. The second, third, and fourth health care devices 110-a-2, 110-a-3, 110-a-4 may also subscribe to certain types of data that other ones of the health care devices publish on the shared data channel. The physical layer defining the shared data channel thus provides a data bus connecting the second, third, and fourth health care devices 110-a-2, 110-a-3, 110-a-4. In addition, however, the server 115 may receive some or all of the data published on the shared data channel and duplicate it on a separate shared data channel and/or store the data in a database for later access by a health care device 110 or a provider 120, 125, 130, 135, 140. In general, the network 105 may include one or more data buses, with each data bus allowing any number of health care devices 110 to publish data on the bus and/or subscribe to data published by other health care devices 110, and, when the network 105 includes multiple data buses, the multiple data buses may optionally cooperate together to provide interconnectivity between the multiple data buses such that the health care devices 110 in essence form a single, shared data space.

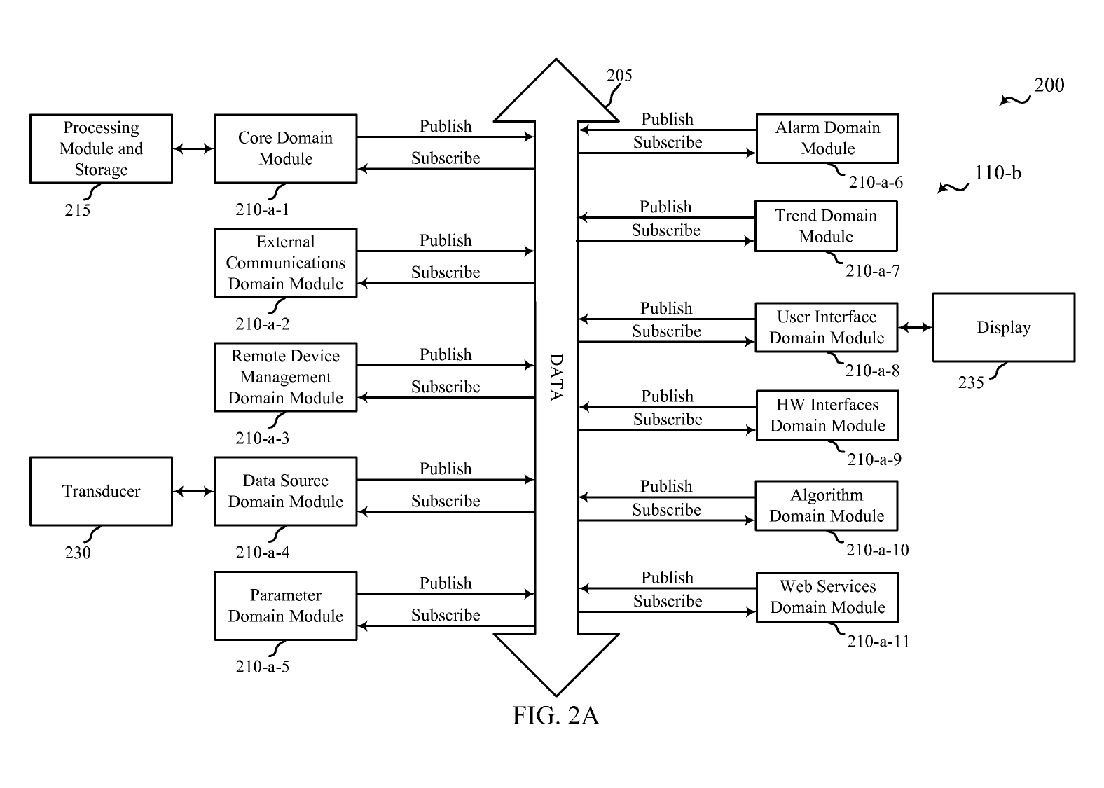

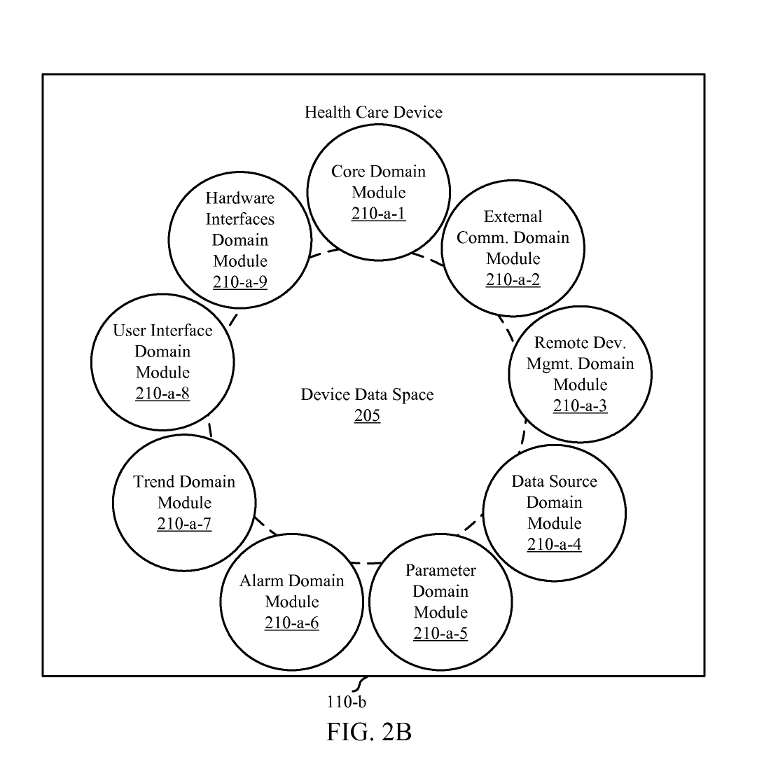

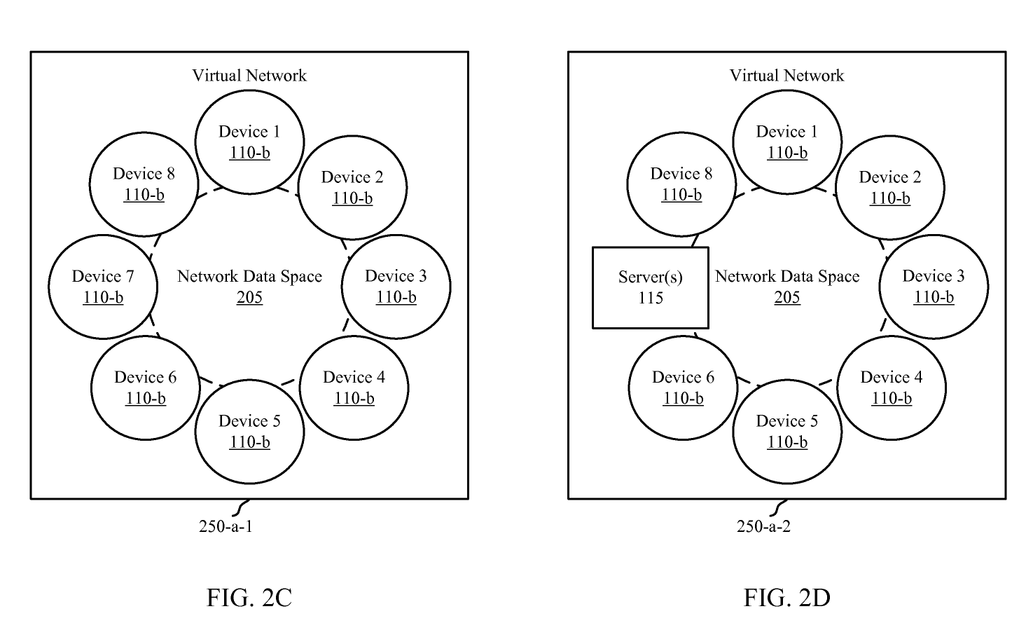

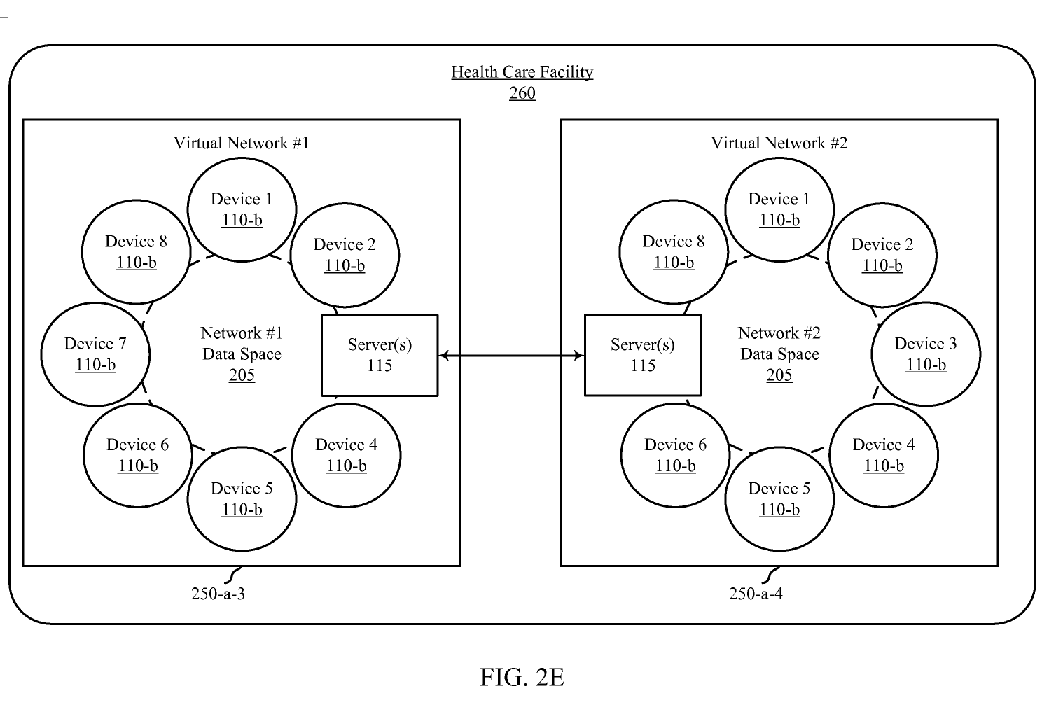

Still referring to the distributed health care monitoring system 100 illustrated in FIG. 1, a number of functions associated with a health care monitoring process may be defined and distributed among two or more of the health care devices 110 shown in FIG. 1. These functions include all of the tasks and responsibilities needed for a comprehensive health care monitoring process, including receiving and processing raw physiological or clinical measurements (such as oxygen level, heart rate, blood pressure, temperature, and so forth), generating parameters interpretable by health care providers based on the raw data, setting and monitoring alarms based on raw measurements or parameters, defining and implementing algorithms based on raw measurements or parameters, communicating among various system components, displaying data, remotely managing health care devices, and so forth. The functions may be defined by one or more of the providers 120, 125, 130, 135, 140, and/or may follow one or more industry standards. The functions may also be defined or refined by, for example, a sustaining engineer. As described below with reference to FIG. 2A, the multiple functions may be implemented by a respective group of monitoring modules, with each of the health care devices 110 in FIG. 1 including one or more of the monitoring modules implementing the defined functions. In some embodiments, at least some of the health care devices 110 are interoperable with multiple types of different health care devices 110, including health care devices 110 made by different manufacturers.

In operation, the health care devices 110 are connected by the network 105 and are utilized collectively to monitor a health care patient. The monitoring of the patient may include carrying out some or all of the defined functions described herein and provide data related to the monitoring to one or more of the providers 120, 125, 130, 135, 140. For example, some or all of the data generated by the health care devices 110 may be provided to a health care provider 120 (such as a doctor or nurse) in real-time or near real-time through the network 105, or, alternatively the data may be stored on server 115 for aggregation and later analysis by a health care provider 120. Also, some or all of the data generated by the health care devices may be provided to the records provider 140, which may be an electronic medical records (EMR) provider. Some or all of the data may also be provided to an equipment provider 130, such as the manufacturer of one or more of the health care devices 110, in order for the equipment provider to determine charges associated with the use of the health care devices 110. Some or all of the data may also be provided to an analysis provider 135 (which in some embodiments may be the same as the equipment provider 130), and the analysis provider 135 may analyze the received data and provide business intelligence to the users of the health care devices, including decision support, trend information, suggested training opportunities, and so forth.

Turning now to the block diagram 200 of a health care device 110-b in FIG. 2A, several examples of monitoring modules 210 are shown. As mentioned above, one or more monitoring modules 210 may be included in each of the health care devices 110 in the health care monitoring data network 105 of FIG. 1, with the monitoring modules 210 implementing respective ones of the defined functions of the health care monitoring process. The health care device 110-b in FIG. 2A may be an example of one or more aspects of the health care devices 110 shown and described above with reference to FIG. 1.

Each module 210 may be implemented with one or more application-specific integrated circuits (ASICs) adapted to perform some or all of the applicable functions in hardware. Alternatively, the functions may be performed by one or more other processing units (or cores), on one or more integrated circuits. In other embodiments, other types of integrated circuits may be used (e.g., Structured/Platform ASICs, Field Programmable Gate Arrays (FPGAs), and other Semi-Custom ICs), which may be programmed in any manner known in the art. The functions of each module 210 may also be implemented, in whole or in part, with instructions embodied in a memory (e.g., as a software module), formatted to be executed by one or more general or application-specific processors.

Each monitoring module 210 may be associated with a “domain” that refers to the defined functionality provided by that monitoring module. As illustrated in FIG. 2A, some of the types of monitoring modules 210 that may be included in any given health care device 110-b include a core domain module 210-a-1, an external communications domain module 210-a-2, a remote device management domain module 210-a-3, a data source domain module 210-a-4, a parameter domain module 210-a-5, an alarm domain module 210-a-6, a trend domain module 210-a-7, a user interface domain module 210-a-8, a hardware interfaces domain module 210-a-9, an algorithm domain module 210-a-10, and a web services domain module 210-a-11, each of which will be described below. Other monitoring modules 210 are also contemplated.

Each health care device, such as the health care devices 110-b shown in FIG. 2A, may include one or more of the monitoring modules 210, but in at least some embodiments, no health care device includes all of the monitoring modules 210. Instead, multiple heath care devices 110 (and their associated monitoring modules 210) may cooperatively work together to accomplish all of the functions associated with the health care monitoring process. In other embodiments, however, all or nearly all of the monitoring modules 210 may be included in a single health care device 110-b, such as that shown in FIG. 2A.

Each of the monitoring modules 210 in FIG. 2A is coupled with a data bus 205 of the health care device 110-b. The monitoring modules 210 may publish certain types of data to the data bus 205 and may also subscribe to certain types of data published on the data bus 205 by other monitoring modules 210. In some embodiments, and as shown in FIG. 2A, the data bus 205 may be a shared physical medium (e.g., wires) within the health care device 110-b itself. In other embodiments, and as described above, the data bus may be defined by the health care monitoring network 105 (e.g., an Ethernet local area network (LAN), or a shared Wi-Fi channel) and a number of health care devices 110 may be coupled therewith. In still other embodiments, a combination of a local data bus 205 within the health care device 110-b and a shared data bus for several health care devices 110 may be used.

A data distribution service (DDS) protocol defining certain types or “topics” of data may be used by the monitoring modules 210 to publish and subscribe to data on the local data bus 205 and/or on the data bus of the network 105. The types of data may include various raw measurements from transducers, parameters, alarms, trend information, algorithm information, graphical representations, control signals, and so forth. Taking the data source domain module 210-a-4 as an example, the data source domain module 210-a-4 may receive raw measurements from a transducer 230 (which may include one or more sensing elements, including electrodes, accelerometers, etc.) coupled with the data source domain module 210-a-4, may format the raw measurements according to the DDS protocol, and then publish (i.e., transmit) the formatted measurements on the data bus 205 if the data bus 205 is available for transmission. The data source domain module 210-a-4 may subscribe to control signals published on the data bus 205, including control signals published by the core domain module 210-a-1. In some embodiments, each transmission of data on the data bus 205 by any of the modules 210 may include an address or other identifier that identifies the source of the data transmission. In other embodiments, no identification of the source of the data transmission is given. Even in those transmissions that do include source identifiers, however, the monitoring modules 210 and the health care devices 110 may be agnostic as to the source of the data in some embodiments.

Although the health care device 110-b shown in FIG. 2A includes several specific monitoring modules 210, it will be appreciated that a health care device 110 may include one or more of the monitoring modules 210, similar or different monitoring modules not shown in FIG. 2A, and so forth. Thus, while several specific monitoring modules 210-a-1, 210-a-2, 210-a-3, 210-a-4, 210-a-5, 210-a-6, 210-a-7, 210-a-8, 210-a-9, 210-a-10, 210-a-11 are shown in FIG. 2A and described below as an example of how the functions of a health care monitoring process may be distributed among a number of monitoring modules, and those monitoring modules may be distributed among a number of health care devices 110, many other combinations and permutations are contemplated.

Referring still to the example shown in FIG. 2A, the core domain module 210-a-1 may be specific to and included in each health care device in some embodiments, and may be responsible for integrating the hardware and its specific functions into the overall health care monitoring system 100. The core domain module 210-a-1 may perform administrative functions, including sending control signals to other modules 210 of the health care device, policing the shared data bus 205, starting up and shutting down the various domain modules on the health care device, maintaining the configuration associated with an application, and so forth. As illustrated in FIG. 2A, the core domain module 210-a-1 may be coupled with a processing module and storage 215, which may include a data processor and data storage. The data processor may process and analyze any data published on the data bus 205, and the data storage may store some, none, or all of the data published on the data bus 205.

The external communications domain module 210-a-2 may be configured to allow the health care device 110-b to interact and communicate with third party health care devices (e.g., health care devices from another manufacturer or health care devices that do not publish and subscribe to the shared data space as described herein). The external communications domain module 210-a-2 may be configured to communicate with the third party health care devices using a proprietary communications protocol, thus providing a translation function between the third party health care device and the data bus 205 and other health care devices 110-b in the system. The external communications domain module 210-a-2 may also subscribe to control signals from the core domain module 210-a-1 sent over the data bus 205, and may publish status information to the data bus 205.

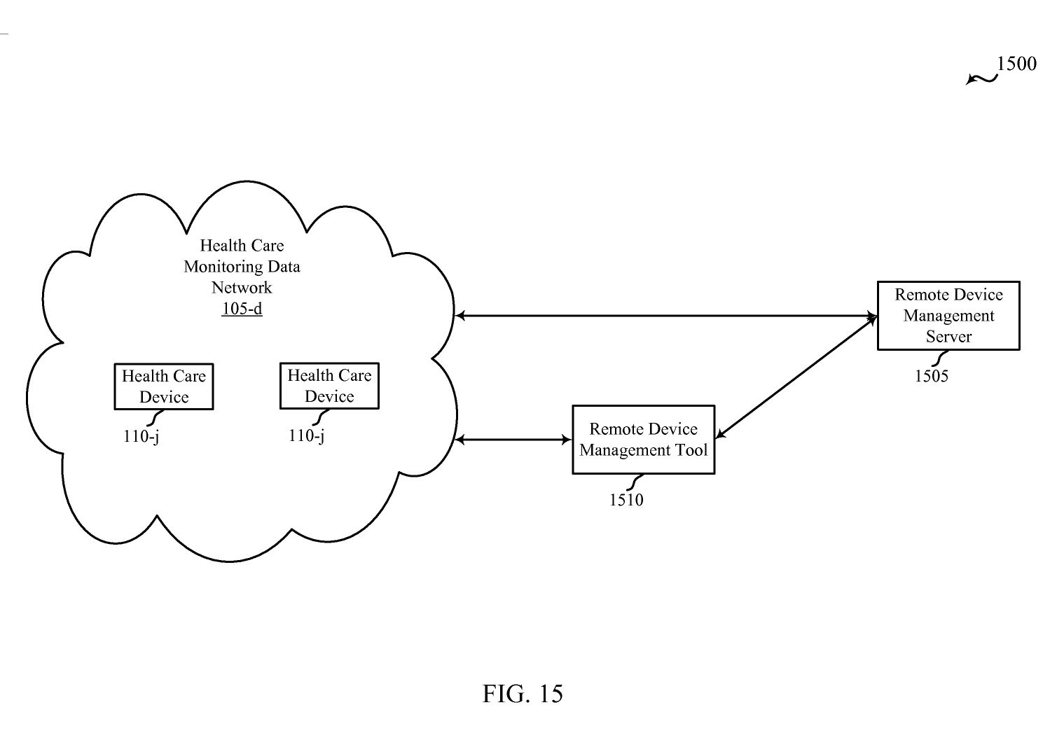

The remote device management domain module 210-a-3 may be configured to couple the health care device 110-b with a remote device management server, and to receive instructions from and/or send information to the remote device management server, which is explained in further detail below with reference to FIGS. 15 through 18. For example, the remote device management domain module 210-a-3 may subscribe to instructions from a remote device management server or tool, and may publish information responsive to the instructions. The remote device management domain module 210-a-3 may also subscribe to control signals from the core domain module 210-a-1 sent over the data bus 205, and may publish status information to the data bus 205.

As mentioned above, the data source domain module 210-a-4 may be configured to supply received data (e.g., from transducer 230) to the other monitoring modules. As such, the data source domain module 210-a-4 may format the received data into packets and then publish the formatted data packets to the data bus 205. The data source domain module 210-a-4 may also subscribe to control signals from the core domain module 210-a-1 sent over the data bus 205, and may publish status information to the data bus 205. Note that while only a single transducer 230 and single data source domain module 210-a-4 are shown in FIG. 2A, in some embodiments, many different transducers may measure many different physiological or clinical conditions, and the different types of measurements may be provided to a single or multiple different data source domain modules 210-a-4.

In some embodiments, the data source domain 210-a-4 may further be configured to synchronize data using common and/or functionally equivalent parameters or raw data (e.g., respiration rate derived from plethysmograph data, impedance data, or capnography data) from separate health care sensor devices. The data source domain 210-a-4 may synchronize these various types of data by recognizing patterns based on the inherent variability of physiologic signals as an alternative to time-stamping, or when time-stamping is not a part of the record. In other embodiments, this synchronization may be provided by the parameter domain module 210-a-5, described next, or some other domain module.

The parameter domain module 210-a-5 may be configured to parse the data measured by the transducer 230 and also to generate one or more parameters that are interpretable by a health care provider 120 based on that data from the transducer 230. As such, the parameter domain module 210-a-5 subscribes to the data published by the data source module(s) 210-a-4, and publishes the parameters generated based on the one or more types of data published by the data source module(s) 210-a-4. The parameter domain module 210-a-5 may also subscribe to control signals from the core domain module 210-a-1 sent over the data bus 205, and may publish status information to the data bus 205.

The alarm domain module 210-a-6 may be configured to generate one or more alarms responsive to the parameters published by the parameter domain module 210-a-5 and/or responsive to the raw data measurements from the data source domain module 210-a-4 exceeding a predetermined threshold. As such, the alarm domain module 210-a-6 subscribes to the raw measurements published by the data source domain module 210-a-4 and/or the parameters published by the parameter domain module 210-a-5, and publishes alarms to the data bus 205. In some embodiments, the alarm domain module 210-a-6 may support dynamic alarm configuration based at least in part on co-related parameters or conditions. The alarm domain module 210-a-6 may also subscribe to control signals from the core domain module 210-a-1 sent over the data bus 205, and may publish status information to the data bus 205.

The trend domain module 210-a-7 may be configured to maintain trends (e.g., summary data) of raw measurements and/or parameters. As such, the trend domain module 210-a-7 subscribes to the raw measurements published by the data source domain module 210-a-4 and/or the parameters published by the parameter domain module 210-a-5, and publishes trend information to the data bus 205. The trend domain module 210-a-7 may also subscribe to control signals from the core domain module 210-a-1 sent over the data bus 205, and may publish status information to the data bus 205.

The user interface domain module 210-a-8 may be configured to graphically display the raw measurements, the parameters, the alarms, the trend information and so forth on a display 235 coupled with the user interface domain module 210-a-8. As such, the user interface domain module 210-a-8 may subscribe to any of the data published on the data bus 205 in order to present a graphical depiction of the received data. The user interface domain module 210-a-8 may also subscribe to control signals from the core domain module 210-a-1 sent over the data bus 205, and may publish status information to the data bus 205.

The hardware interfaces domain module 210-a-9 may be configured to encapsulate the various hardware idiosyncrasies associated with the health care device 110-b. In this regard, the hardware interfaces domain module 210-a-9 provides a hardware abstraction layer, at which an operating system or application platform can interact with the health care device 110-b at a general or abstract level rather than at a detailed hardware level. The hardware interfaces domain module 210-a-9 allows one or more business processes or functionality (e.g., such as processes associated with the parameter domain module 210-a-5, the alarm domain module 210-b-6, and the user interface domain module 210-a-8) to make a call to the hardware interfaces of the health care device 110-b in a manner that can be agnostic with regard to how the call is implemented on various operating systems or platforms. For example, the alarm domain module 210-b-6 can maintain the same call, code, and/or logic to generate an alarm call, and the hardware interfaces domain module 210-a-9 can maintain and implement the requirements for how a particular alarm (e.g., an alarm tone) is to be generated. As such, the hardware interfaces domain module 210-a-9 can receive multiple instances of the same alarm from the alarm domain module 210-b-6 and provide each respective instance to different operating systems or platforms.

In some examples, the hardware interfaces domain module 210-a-9 may subscribe to control signals from the core domain module 210-a-1, the parameter domain module 210-a-5, the alarm domain module 210-b-6, and the user interface domain module 210-a-8 sent over the data bus 205, and may publish status information to the data bus 205. Additionally or alternatively, in certain implementations, the hardware interfaces domain module 210-a-9 (or instances thereof and/or like hardware interfaces domain modules) can be on a different hardware interface or device than a domain module that is generating a particular alarm. For example, a server may generate an alarm (e.g., using alarm domain module techniques described herein) that is received by a tablet computer having a hardware interfaces domain module (e.g., using an instance of the hardware interfaces domain module 210-a-9 and/or the hardware domain module techniques described herein).

The algorithm domain module 210-a-10 may be configured to implement one or more algorithms, which may provide analysis and interpretation of certain types of data (including raw measurements, parameters, alarms, etc.). The algorithms may in some instances be user-defined or may be contained within third-party applications obtained or purchased from an application store. As such, the types of data that the algorithm domain module 210-a-10 subscribes to and the types of the data that the algorithm domain module 210-a-10 publishes may be highly specific to which algorithm is being executed by the algorithm domain module 210-a-10 and the correlation of the monitored parameters. The algorithm domain module 210-a-10 may, in any event, subscribe to control signals from the core domain module 210-a-1 sent over the data bus 205, and may publish status information to the data bus 205.

The web services domain module 210-a-11 may be configured to provide web services to the health care device 110-b. For example, the web services domain module 210-a-11 may provide software allowing interaction of the health care devices 110-b over the network via certain predefined protocols and signaling mechanisms—for example using Extensible Markup Language (XML) or hypertext transfer protocol (HTTP). The web services domain module 210-a-11 may also subscribe to control signals from the core domain module 210-a-1 sent over the data bus 205 and may publish status information to the data bus 205.

In some embodiments, various providers may design and implement custom algorithms that suit their particular practice and contribution to the health care monitoring process. Some examples of algorithms include algorithms related to certain aspects of ventilation, fluid management, autoregulation, intensive care monitoring, delivery room monitoring, and so forth.

Referring still to FIGS. 1 and 2A, in some embodiments, the system 100 may be extended to also allow third party health care devices (e.g., proprietary health care devices of another company, health care devices that are not architected as described herein, etc.) to communicate with the health care devices 110 in FIGS. 1 and 2 via the data bus 205. These third party health care devices may communicate using a proprietary or standardized protocol, and the system 100 may include the external communications domain module 210-a-2 described above, which may be configured to interface with the data bus 205 and the health care devices 110 on behalf of the third party health care device. In this manner, the external communications domain module 210-a-2 may intercept data available on the data bus 205 and provide the same to the third party health care device, may store or rebroadcast data from the third party health care device on the data bus 205 in the appropriate format such that the data can be used and interpreted by other health care devices coupled to the data bus 205.

In some embodiments, the system 100 may provide a configurable rules engine, by which a user may create logical relationships between domains and between independent variables or elements within a domain in order to produce an index or flag for indicating a predefined condition has been met. Also, in some embodiments, the system 100 may be adaptable and/or resilient (similar to neural net training) in that it may create domain modules that track and/or learn use and behavior patterns and device use preferences. The adaptable/resilient system may also be able to weigh based on other results and criteria that are derived from a central data repository. It is also contemplated that the system 100 may be responsive to instantaneous and/or transient data (e.g., a new parameter or functionality) being introduced into the shared data space and be able to dynamically enhance or reduce the health care monitoring based on the discovered changes. Additionally, associated hierarchal rules used to govern the data space are contemplated, whereby the priority of parameters, their associated states and messages, including alarms and/or the display of features, may be conditional or specific to the location and/or the monitored demographics of a patient.

FIG. 2B shows another block diagram of the health care device 110-b from FIG. 2A, with each of a plurality of domain modules 210-a-1, 210-a-2, 210-a-3, 210-a-4, 210-a-5, 210-a-6, 210-a-7, 210-a-8, 210-a-9 sharing a device data space 205 (which may be an example of one or more aspects of the data bus 205 in FIG. 2A.

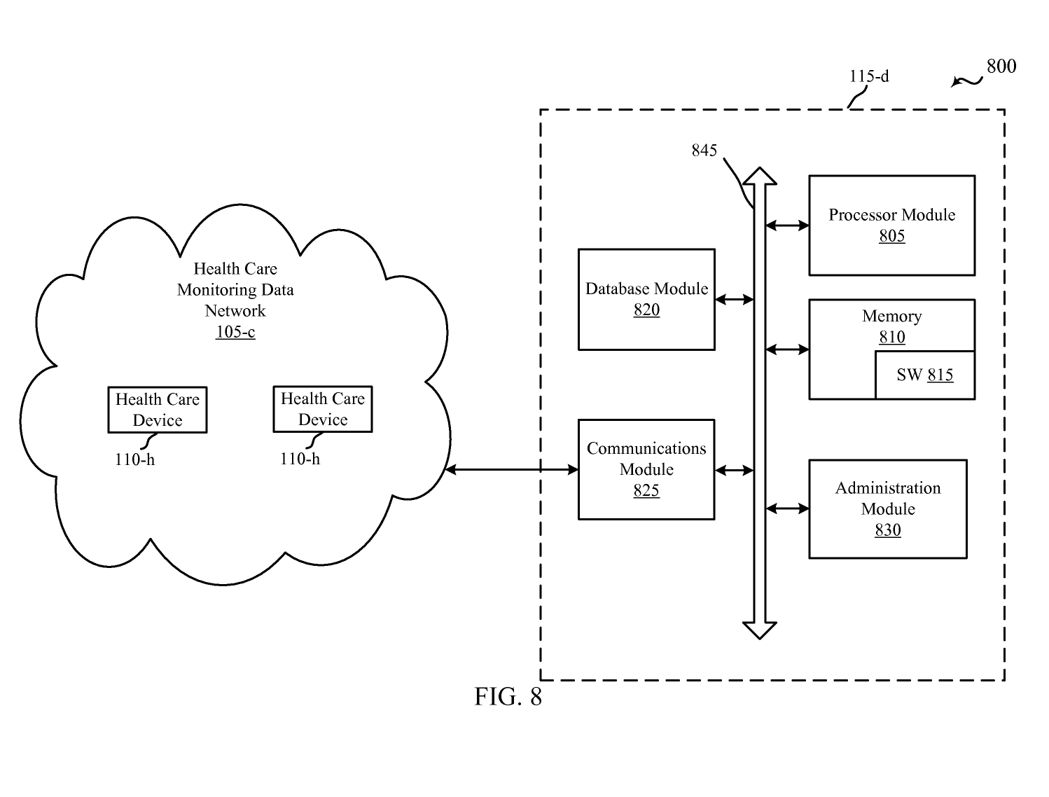

FIGS. 2C and 2D show examples of virtual networks 250-a-1, 250-a-2, each with a plurality of health care devices 110-b. FIG. 2C, in particular, shows a virtual network formed of eight health care devices 110-b, but with no server. In this manner, the health care devices 110-b communicate directly with one another (e.g., via the network data space 205) to administer the health care monitoring functions. FIG. 2D, on the other hand, includes a number of health care devices 110-b and also includes one or more servers 115 that also share the network data space 205 with the health care devices 110-b. The one or more servers 115 may provide the functionality described above with reference to FIGS. 1 and 2A and described below with reference to FIG. 8. In any event, and as shown in FIGS. 2C and 2D, many different network architectures are contemplated, including those with and without centralized management servers.

FIG. 2E shows a block diagram of how many different health care devices 110-b may be interconnected within a health care facility 260. As shown in FIG. 2E, the health care facility may include two or more virtual networks 250-a-3, 250-a-4, each of which includes a number of health care devices 110-b, one or more servers 115, and a respective network data space 205. The servers 115 from each virtual network 250-a-3, 250-a-4 may be interconnected (e.g., by a shared data bus defining a shared data space between the two virtual networks 250-a-3, 250-a-4), which may allow inter-network communication between the health care devices 110-b.

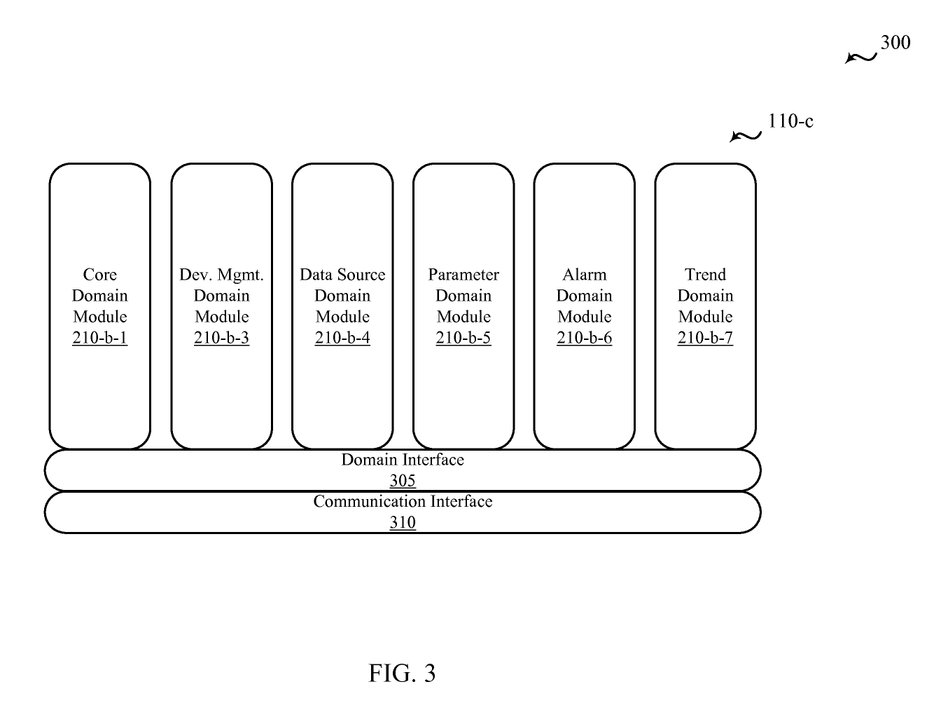

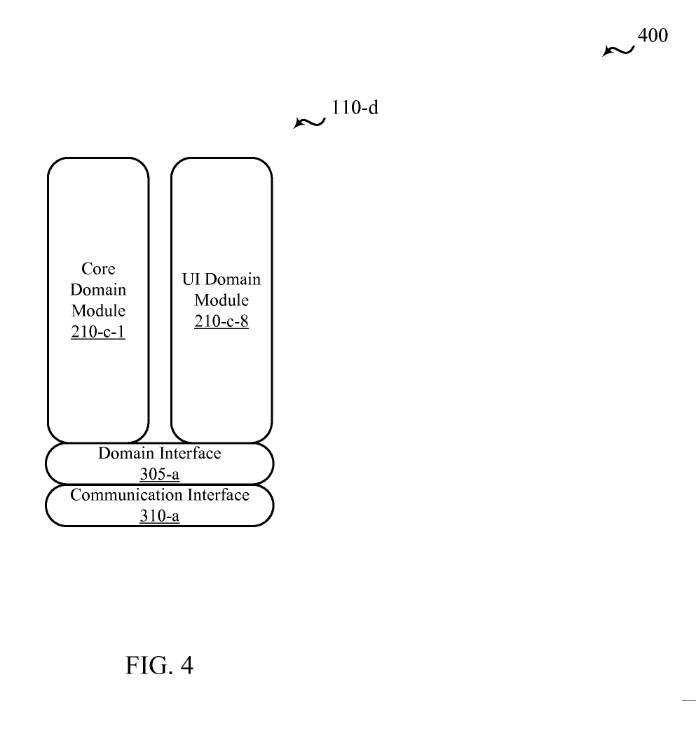

Turning now to FIG. 3 and FIG. 4, examples are given of health care devices 110-c, 110-d each incorporating only a subset of the monitoring modules 210 shown and described with reference to FIG. 2A. The health care devices 110-c, 110-d and their associated monitoring modules 210 may be examples of one or more aspects of the health care devices 110 and the monitoring modules 210 shown and described above with reference to FIGS. 1 and 2. As mentioned above, in some embodiments, the defined functions associated with the health care monitoring process may be distributed among two or more health care devices 110, and FIGS. 3 and 4 illustrate one example of how two different health care devices 110-c, 110-d may work together to perform a health care monitoring process.

Each health care device 110-c, 110-d in FIGS. 3 and 4, and their respective monitoring modules 210, may be implemented with one or more application-specific integrated circuits (ASICs) adapted to perform some or all of the applicable functions in hardware. Alternatively, the functions may be performed by one or more other processing units (or cores), on one or more integrated circuits. In other embodiments, other types of integrated circuits may be used (e.g., Structured/Platform ASICs, Field Programmable Gate Arrays (FPGAs), and other Semi-Custom ICs), which may be programmed in any manner known in the art. The functions of each health care device 110 or monitoring module 210 may also be implemented, in whole or in part, with instructions embodied in a memory (e.g., as a software module), formatted to be executed by one or more general or application-specific processors.

Turning now specifically to FIG. 3, an example of a health care device 110-c is shown. The health care device 110-c in FIG. 3 includes a core domain module 210-b-1, a remote device management domain module 210-b-3, a data source domain module 210-b-4, a parameter domain module 210-b-5, an alarm domain module 210-b-6, and a trend domain module 210-b-7, which all may be examples of aspects of the respective monitoring modules 210 shown and described with reference to FIG. 2A. The health care device 110-c in FIG. 3 also include a domain interface 305, which may be an example of aspects of the data bus 205 in FIG. 2A and may allow communication among the monitoring modules 210-b-1, 210-b-2, 210-b-3, 210-b-4, 210-b-5, 210-b-6, 210-b-7 of the health care device 110-c. The domain interface 305 may include an associated data dictionary that allows independent elements and/or results produced from separate data sources to be correlated by structure, thus allowing the combination of all data to reveal patterns for recognition. The data dictionary may also provide an interface to define behavior (e.g., of user interface components). The health care device 110-c in FIG. 3 also includes a communication interface 310, which may be configured to allow communication with other health care devices (e.g., the health care device 110-d in FIG. 4) over the network 105. The communication interface 310 may in some embodiments provide an interface between the data bus 205 of the individual health care device 110-b and the data bus of the network 105 so that published data flows between the local data bus 205 and the network's data bus. The health care device 110-c in FIG. 3 does not, however, include a user interface domain module or a display.

Turning next, however, to FIG. 4, an example of a health care device 110-d is shown that does have a user interface domain module and a display, and that can thus provide the display functionality to a health care provider monitoring a patient. More specifically, the health care device 110-d in FIG. 4 includes a core domain module 210-c-1 and a user interface domain module 210-b-8, which all may be examples of aspects of the respective monitoring modules 210 shown and described with reference to FIG. 2A. The user interface domain module 210-c-8 may be coupled with a display (not shown in FIG. 4). The health care device 110-d in FIG. 4 also include a domain interface 305-a, which may be an example of aspects of the domain interface 305 in FIG. 3. The health care device 110-d in FIG. 4 also includes a communication interface 310-a, which may be an example of aspects of the communication interface 310 in FIG. 3. The health care device 110-d in FIG. 4 does not, however, include a data source domain module, a parameter domain module, an alarm domain module, and so forth.

Even though the health care devices 110-c, 110-d shown in FIGS. 3 and 4 do not individually include all of the monitoring modules 210 shown in FIG. 2A and hence do not individually implement all of the defined functions of the health care monitoring process, the health care devices 110-c, 110-d are connected via the network 105 and can, together, provide a comprehensive health care monitoring system. In one example, the health care device 110-c in FIG. 3 may be a bedside health care device with one or more transducers, but without any display. The health care device 110-d in FIG. 4 may be a tablet computer carried by a health care provider 120, such as a doctor or a nurse, but that does not include any transducers or any processing equipment. It will be appreciated that because at least one of the functions associated with the health care monitoring process (e.g., displaying data via a user interface) has been decoupled from other functions (e.g., collecting and processing physiological or clinical measurements), these decoupled, different functions can be distributed among at least two different health care devices 110-c, 110-d. Similarly, it will be appreciated that by decoupling the data from specific, complex health care devices and instead publishing it to the common data bus 205, many different components may be able to constructively work together in the health care monitoring process.

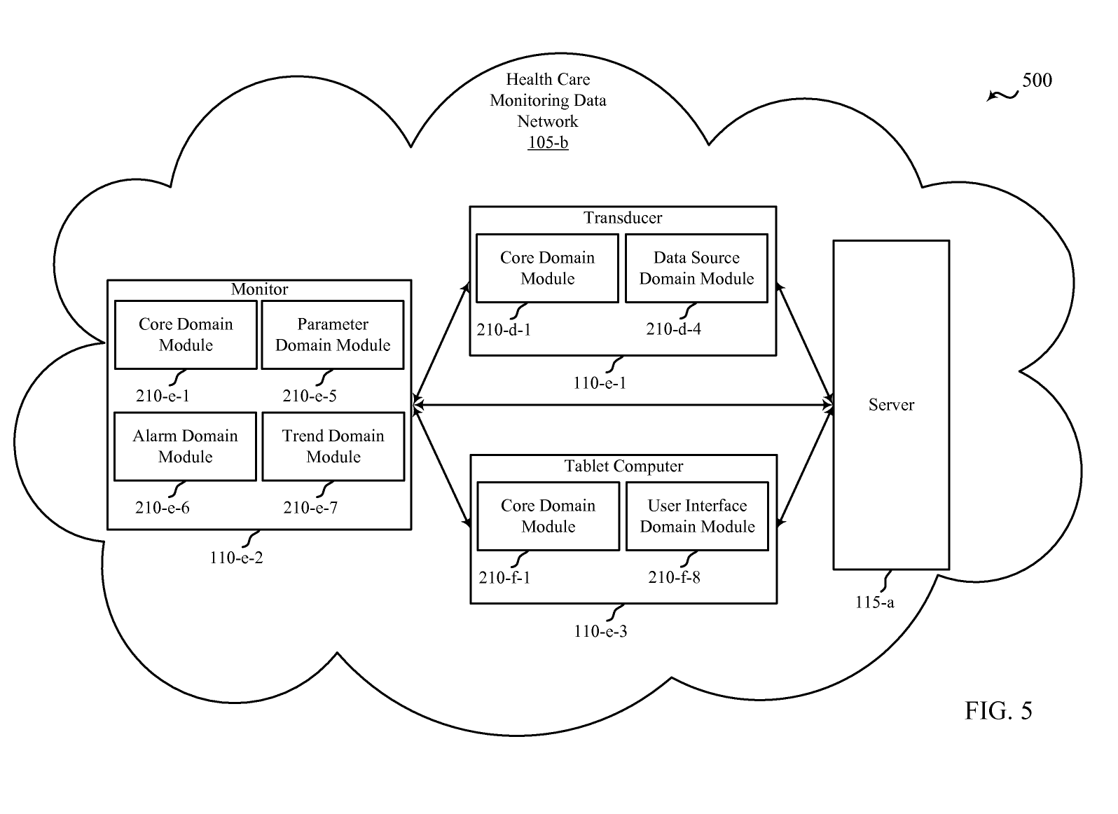

FIG. 5 is a block diagram illustrating a distributed health care monitoring system 500 in which, as in FIGS. 3 and 4, the functions of a health care monitoring process are distributed among multiple health care devices 110-e-1, 110-e-2, 110-e-3. In the system 500 in FIG. 5, the health care devices are labeled in as a transducer health care device 110-e-1, a monitor health care device 110-e-2, and a tablet computer health care device 110-e-3, each of which may be directly and/or indirectly coupled with the server 115-a via the network 105-a. The system 500, including the health care devices 110-e-1, 110-e-2, 110-e-3, the server 115-a, and the network 105-b may be examples of aspects of the respective system 100, health care devices 110, and server 115 shown and described above with reference to FIG. 1.

Each health care device 110-e-1, 110-e-2, 110-e-3 in FIG. 5, and their respective monitoring modules 210, as well as the server 115-a may be implemented with one or more application-specific integrated circuits (ASICs) adapted to perform some or all of the applicable functions in hardware. Alternatively, the functions may be performed by one or more other processing units (or cores), on one or more integrated circuits. In other embodiments, other types of integrated circuits may be used (e.g., Structured/Platform ASICs, Field Programmable Gate Arrays (FPGAs), and other Semi-Custom ICs), which may be programmed in any manner known in the art. The functions of each health care device 110, monitoring module 210, and the server 115-a may also be implemented, in whole or in part, with instructions embodied in a memory (e.g., as a software module), formatted to be executed by one or more general or application-specific processors.

The transducer health care device 110-e-1 in FIG. 5 includes a core domain module 210-d-1 and a data source domain module 210-d-4, along with a transducer (not shown in FIG. 5) coupled with the data source domain module 210-d-4. The monitor health care device 110-e-2 in FIG. 5 includes a core domain module 210-e-1, a parameter domain module 210-e-5, an alarm domain module 210-e-6, and a trend domain module 210-e-7. The tablet computer health care device 110-e-3 in FIG. 5 includes a core domain module 210-f-1, and a user interface domain module 210-f-8. In operation, the transducer health care device 110-e-1 receives physiological or clinical measurements via the data source domain module 210-d-4, and may publish those measurements to the monitor health care device 110-e-2 and the tablet computer health care device 110-e-3 directly over the network 105-a (e.g., using a wireless connection between the transducer health care device 110-e-1 and the tablet computer health care device 110-e-3), or may publish those measurements to the server 115-a, which then transmits the measurements to the monitor and/or tablet computer health care devices 110-e-2, 110-e-3.

Referring still to the system 500 shown in FIG. 5, in some embodiments, some of the defined health care monitoring functions (e.g., those associated with the parameter domain module 210-e-5, the alarm domain module 210-e-6, the trend domain module 210-e-7, and so forth) may be performed by the server 115-a or other hardware on the network 105-b other than the health care devices themselves. In this manner, only some of the defined health care monitoring functions may be performed by the health care devices. For example, and still referring to FIG. 5, in one embodiment, the monitor health care device 110-e-2 may be excluded and its functionality replaced by the server 115-a. In this configuration, the tablet computer health care device 110-e-3 does very little other than communicate with other health care devices and provide a display, and the transducer health care device 110-e-1 does very little other than receive raw measurements and format them for publishing on the network 105-b. Many of the defined health care monitoring functions can thus be provided by the infrastructure of the network 105-b, which may reduce the cost of the health care devices (including maintenance, training, repair, and replacement of the devices) and of providing the overall health care monitoring process.

Referring now to FIGS. 3 through 5, in some embodiments, each health care device 110 may include at least a core domain module and one additional monitoring module configured to perform one of the defined functions associated with the health care monitoring process. In other embodiments, however, some devices may not include even these monitoring modules.

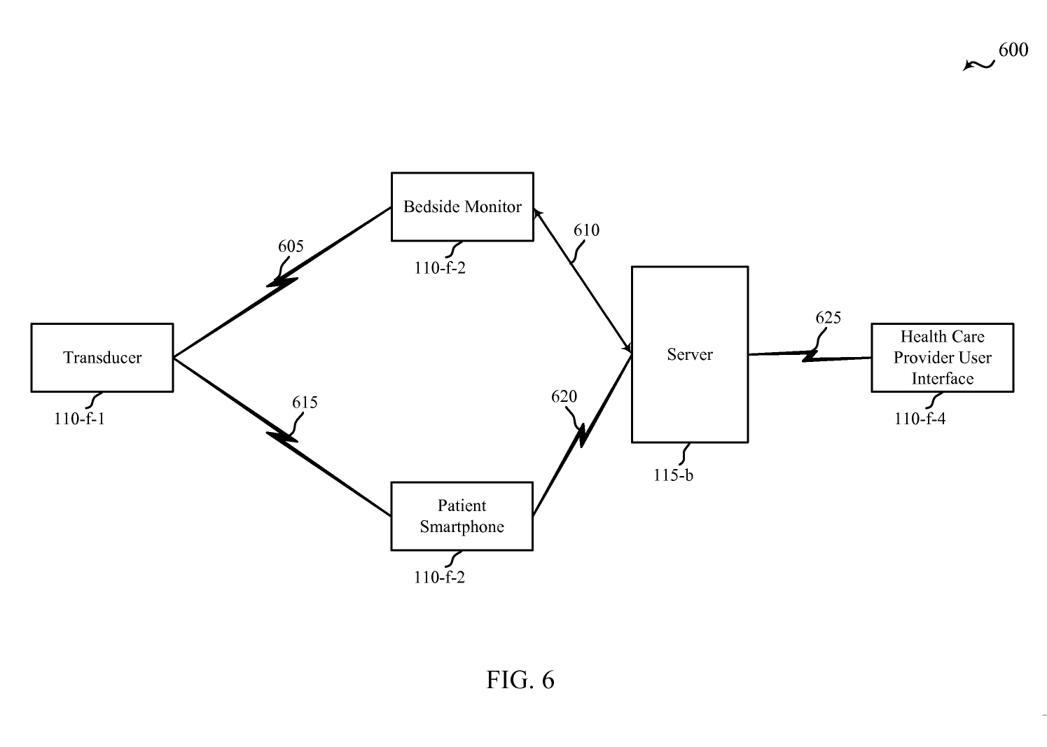

FIG. 6 is a block diagram of another example of a distributed health care monitoring system 600, which includes a transducer health care device 110-f-1, a bedside monitor health care device 110-f-2, a patient smartphone (which can be used as a health care device) 110-f-2, a server 115-b, and a health care provider user interface health care device 110-f-4. The health care devices 110-f-1, 110-f-2, 110-f-3, 110-f-4 and the server 115-b may be examples of one or more aspects of the health care devices 110 and servers 115 shown and described above with reference to FIGS. 1 through 5. The system 600 shown in FIG. 6 illustrates how the health care devices 110 and monitoring modules 210 described above can be used to dynamically switch between various health care devices to provide one or more monitoring functions.

Each health care device 110-f-1, 110-f-2, 110-f-3, 110-f-4 and their respective monitoring modules, as well as the server 115-b may be implemented with one or more application-specific integrated circuits (ASICs) adapted to perform some or all of the applicable functions in hardware. Alternatively, the functions may be performed by one or more other processing units (or cores), on one or more integrated circuits. In other embodiments, other types of integrated circuits may be used (e.g., Structured/Platform ASICs, Field Programmable Gate Arrays (FPGAs), and other Semi-Custom ICs), which may be programmed in any manner known in the art. The functions of each health care device 110 and the server 115-b may also be implemented, in whole or in part, with instructions embodied in a memory (e.g., as a software module), formatted to be executed by one or more general or application-specific processors.

In the example shown in FIG. 6, the transducer 110-f-1 may be a portable transducer attached to and carried by a patient. The transducer 110-f-1 may be wirelessly coupled with the bedside monitor 110-f-1 via a wireless connection 605 such as Bluetooth, Wi-Fi, Mesh, Thread wireless protocol, Near Field Communication (NFC), etc. In this configuration, the bedside monitor 110-f-2 may be coupled with the server 115-b via a wired connection 610, such as Ethernet or the Internet, and the health care provider user interface 110-f-4 may be wirelessly coupled with the 115-b via wireless connection 625 such as Wi-Fi or a data connection over a cellular telephone network. Again, in this configuration, the bedside monitor 110-f-2 may provide functions associated with a data source domain, a parameter domain, an alarm domain, a trend domain, a hardware interfaces domain, an algorithm domain, and so forth, as described above. However, upon the occurrence of a predetermined event, however, the bedside monitor 110-f-2 may no longer be able to provide one or more of those functions. For example, the power to the bedside monitor 110-f-2 may be lost due to a power outage. In this example, the system 600 may be configured to dynamically switch from having the bedside monitor 110-f-2 provide the monitoring functions to having the patient's smartphone 110-f-2 provide one or more of the monitoring functions. If this scenario, the transducer 110-f-1 may be wirelessly coupled with the patient's smartphone 110-f-2 via wireless connection 615, which may be wirelessly coupled with the server 115-b via wireless connection 620.

FIG. 6 thus shows an example in which a first health care device (i.e., the bedside monitor 110-f-2) is utilized during a first time period to implement a first monitoring function, and a second health care device (i.e., the patient smartphone 110-f-2) is utilized during a second time period to implement the first monitoring function upon, with the switching from utilizing the first health care device to utilizing the second health care device happening upon the occurrence of a pre-defined event. The switching to the second health care device may happen automatically upon the occurrence of the pre-defined event, or the second health care device may notify the user of the occurrence of the pre-defined event and request permission to assume the monitoring function(s) of the first health care device. In some embodiments, the second health care device (i.e., the patient smartphone 110-f-2) may not be utilized during the first time period to implement the monitoring function(s) and similarly the first health care device (i.e., the bedside monitor 110-f-1) may not be utilized during the second time period to implement the monitoring function(s). There may nonetheless be some cases where two separate health care devices implement similar monitoring functionalities at the same time. Also, and as shown in FIG. 6, a separate health care device (i.e., the transducer 110-f-1 in FIG. 6) may implement a separate monitoring function (i.e., providing data source domain functionality) throughout both the first and second time periods. In this manner, the health care devices 110 may dynamically be switched out in certain cases in order to provide continuous or near-continuous monitoring functions.

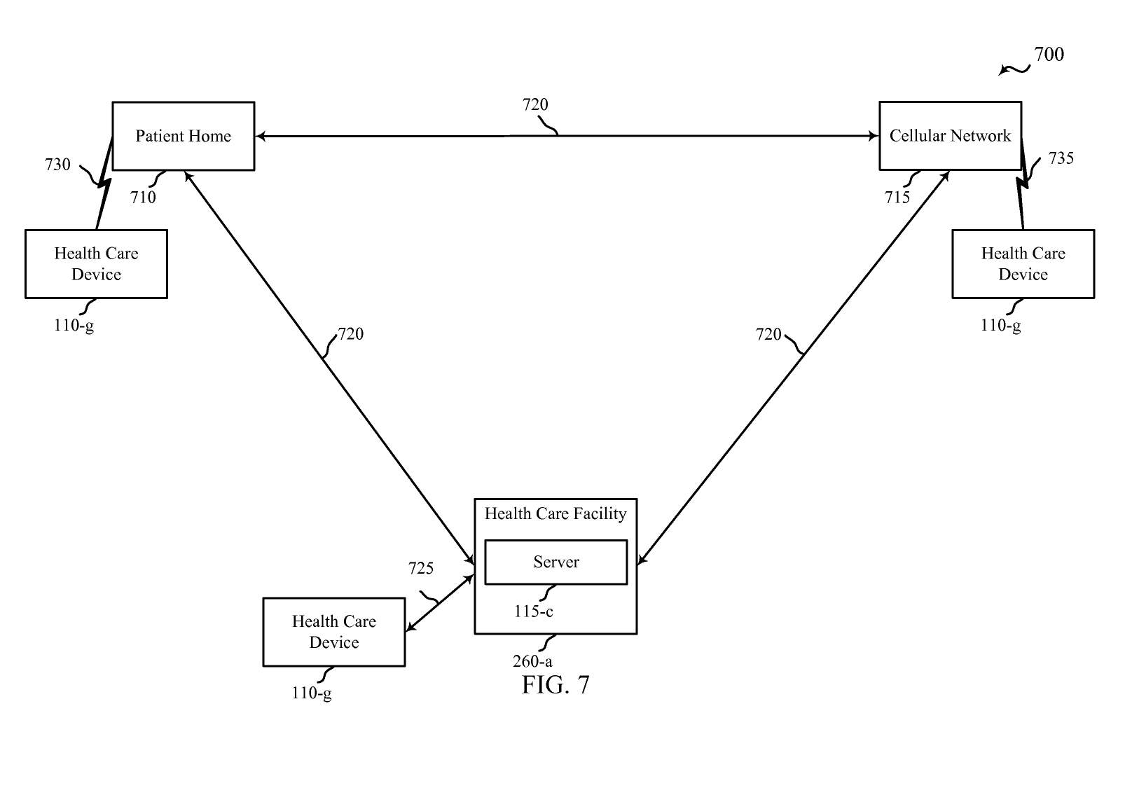

FIG. 7 is a block diagram of another example of a distributed health care monitoring system 700, which includes a health care device 110-g that moves among a number of locations—for example from a health care facility 260-a (which may include a server 115-c storing relevant health care monitoring information), to a patient's home 710, to a cellular data network 715. The cellular phone network 715 may be any location outside of the health care facility 260-a and the patient's home 710 where a wireless wide area network cellular data connection is available. The health care device 110-g and the server 115-c may be examples of one or more aspects of the health care devices 110 and servers 115 shown and described above with reference to FIGS. 1 through 6.

The health care devices 110-g in FIG. 7, as well as the server 115-c, may be implemented with one or more application-specific integrated circuits (ASICs) adapted to perform some or all of the applicable functions in hardware. Alternatively, the functions may be performed by one or more other processing units (or cores), on one or more integrated circuits. In other embodiments, other types of integrated circuits may be used (e.g., Structured/Platform ASICs, Field Programmable Gate Arrays (FPGAs), and other Semi-Custom ICs), which may be programmed in any manner known in the art. The functions of the health care device 110-g and the server 115-c may also be implemented, in whole or in part, with instructions embodied in a memory (e.g., as a software module), formatted to be executed by one or more general or application-specific processors.

As illustrated in FIG. 7, the health care device 110-g may be used to monitor a health care patient and may be coupled with the health care facility 260-a via a wired connection 725 at first. In this configuration, the heath care device 110-g may publish relevant monitoring data to the server 115-c and/or to other health care devices and providers on a network associated with the health care facility 260-a, and may subscribe to data from other health care devices and providers via the network as well. At some point in time, however, the patient may leave the health care facility 260-a to return to his or her home 710. As shown in FIG. 7, the same health care device 110-g may be used to continue monitoring the patient at his or her home 710 if a wireless local area network (WLAN) connection 730, for example, is available. In this configuration, data generated and published by the health care device 110-g may be transmitted to the health care facility 260-a and server 115-c using an Internet connection 720. Similarly the health care device 110-g at the patient's home 710 may subscribe to certain types of data generated and published at the health care facility 260-a using the Internet connection 720.

FIG. 7 also shows a configuration in which the health care device 110-g is taken outside of both the health care facility 260-a and the patient's home 710, but remains within range of a wireless wide area network (WWAN) cellular data network 715. This may represent the movement of a patient and the health care device 110-g in a car, at a store or a park, etc. In this embodiment, the health care device 110-g may be wirelessly coupled with the cellular data network 715 via wireless connection 735, which may connect the health care device 110-g to the health care facility 260-a and server 115-c and/or the patient's home 710 via Internet connections 720 so that the health care device 110-g can publish and/or subscribe to certain types of data within the system 700.

FIG. 7 thus illustrates that the distributed nature of the health care devices 110 described above may allow greater continuity in health care monitoring—including before, during, and after patient visits to health care facilities, with the monitoring data generated by such monitoring following the patient and/or being collected at a central location (e.g., the records providers 140 in FIG. 1).

FIG. 8 shows a diagram 800 of a server 115-d for use with a distributed health care monitoring system, such as the systems 100, 500, 600, 700 described above with reference to FIGS. 1 through 7. The server 115-d in FIG. 8 is coupled with a health care monitoring network 105-c, which may include one or more health care devices 110-h. In some embodiments, the server 115-d may be positioned proximate the health care devices 110-h (e.g., within the same health care facility), whereas in other embodiments the server 115-d may be in a remote location from the health care devices 110-h but coupled therewith through a network connection.

The server 115-d shown in FIG. 8 includes a processor module 805, memory 810 (including software (SW) 715), a database module 820, a communications module 825, and an administration module 830, which each may communicate, directly or indirectly, with each other (e.g., via one or more buses 845). The memory 810 may include random access memory (RAM) and/or read-only memory (ROM). The memory 810 may store computer-readable, computer-executable software/firmware code 815 containing instructions that are configured to, when executed, cause the processor module 805 to perform various functions described herein. Alternatively, the software/firmware code 815 may not be directly executable by the processor module 805 but be configured to cause a computer (e.g., when compiled and executed) to perform functions described herein. The processor module 805 may include an intelligent hardware device, e.g., a central processing unit (CPU), a microcontroller, an application-specific integrated circuit (ASIC), etc. may include random access memory (RAM) and read-only memory (ROM).

The database module 820 of the server 115-d shown in FIG. 8 may be configured to store relevant monitoring information, such as some or all of the data that the health care devices 110-h publish to the data bus of the network 105-c. The database module 820 may store this data for a certain period of time, may summarize the received data and provide a summary to a provider, may analyze the received data, and so forth. The communications module 825 may perform functions related to coupling the server 115-d to the network 105-c, the health care devices 110-h, any relevant providers (not shown in FIG. 8), and so forth, including listening for data that is published by the health care devices 110-h, responding to requests for information from providers, and so forth. The administration module 830 may perform administrative functions regarding the monitoring data received and transmitted using the communications module 825 and monitoring data stored by the database module 820, including administering privacy policies, permissions policies, and so forth.

In some embodiments, the administration module 830 may be configured to dynamically allocate resources to particular tasks based on the availability of different components, modules, domains, and/or personnel. For example, if an algorithm is running only periodically to conserve power on a wearable health care device, but the administration module 830 detects that another health care device with less sensitivity to power consumption becomes available, the administration module 830 may move the responsibility for running the algorithm from the low-power wearable health care device to the newly detected health care device. This switch in functional responsibility may be seamless in that the reporting of the algorithm processing is continuous in some examples. Further, the switch may allow the power consumption of the low-power device to be further reduced while at the same time allowing the algorithm to be run with a greater frequency. As another example, if the limited size of a tablet computer screen is constraining how much data can be displayed, upon the detection of a larger or additional monitor, the administration module 830 may either show additional data on the larger monitor, or switch from using the small, tablet for viewing the information to only using the larger monitor for viewing the information. In this manner, the administration module 830 may be configured to adapt a certain set of health care devices to each particular situation by, for example, minimizing a cost function, maximizing a usefulness function, and so forth.

Referring still to the administration module 830, in some embodiments, the administration module 830 may be configured to dynamically reallocate functionality to different health care devices based on changes in location, device proximity, shift changes for health care workers, admit/discharge status of patients, therapy intervention, alarm or alert conditions, parameter values, trends, and so forth. As one example, the sensor data that is displayed on a health care worker's mobile display may change based on the health care worker's proximity to various patients (e.g., when the health care worker walks into a patient's room, the display may switch to health care information regarding that patient). As another example, an audio device may signal an alarm based on a health care worker's location, assignment, or absence of alarm acknowledgment. As another example, the routing of sensor data to a display may be based on health care worker shift changes. As another example, the frequency at which certain parameters are calculated or certain data is acquired/displayed may be increased or decreased based on the condition of a patient (e.g., a sudden increase in heart rate may trigger an increased heart rate monitoring frequency, whereas a long period of stable heart rate may trigger a decreased heart rate monitoring frequency. As still another example, certain conditions or changes in conditions (e.g., patient condition or initiation or change of therapy) may trigger certain alarms, displays, or parameter calculations—for example, a declining blood oxygen saturation may trigger allocation of display functionality to a respiratory health care provider, or a change in infusion pump settings may trigger allocation of display functionality to an anesthesiologist health care provider.

Further, it will be appreciated that the functions described above with reference to the administration module 830 may be implemented by individual health care devices, or groups of health care devices—e.g., in those embodiments without a centralized server. If, for example, a group of health care devices form an ad hoc network to share data amongst themselves, one of the health care devices may assume the functionality described above related to the administration module, or the functionality may be distributed among several or all of the health care devices in the ad hoc network.

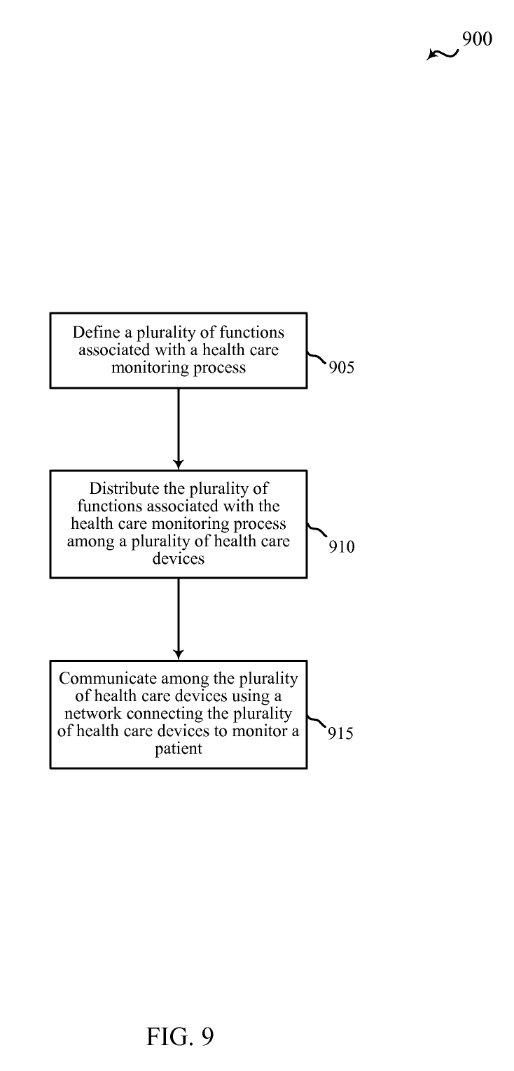

FIG. 9 is a flow chart illustrating an example of a method 900 for health care monitoring in accordance with various aspects of the present disclosure. For clarity, the method 900 is described below with reference to aspects of one or more of the systems 100, 500, 600, 700, health care devices 110, and monitoring modules 210 described above with reference to FIGS. 1-8.

At block 905, the method 900 may include defining a number of functions associated with a health care monitoring process. The functions may be defined by, for example, the equipment providers, the health care providers, the servers, a standards or other industry group or association, or some combination thereof.

At block 910, the method 900 may include distributing the functions associated with the health care monitoring process among multiple health care devices. The functions may be distributed among the health care devices by, for example, the equipment providers when the health care devices are manufactured, or by users of the health care devices during use (e.g., the functions may either be statically distributed to the health care devices with no option to later change them, or they may be dynamically changed while in service as different monitoring needs arise and different equipment becomes available). The functions may be distributed among the health care devices in that monitoring modules implementing the respective functions may be included in respective ones of the health care devices.

At block 915, the method 900 may include communicating among the health care devices using a network connecting the health care devices to monitor a patient. The communications among the health care devices may include the monitoring modules publishing and subscribing to monitoring data, as described above with reference to FIG. 2A. The health care devices may autonomously communicate using the network (e.g., without direct and contemporaneous control by a user such as a health care provider) in some embodiments, whereas in other embodiments, at least some aspects of the publishing and subscribing to monitoring data by the health care devices may be performed in response to a request by a user of the health care devices, such as a health care provider.

It should be noted that the method 900 is just one implementation and that the operations of the method 900 may be rearranged or otherwise modified such that other implementations are possible.

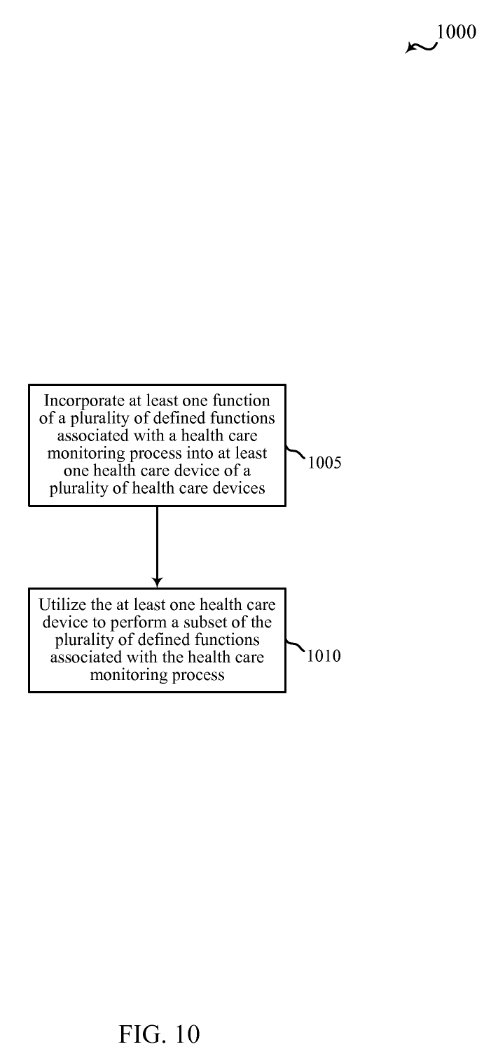

FIG. 10 is a flow chart illustrating an example of a method 1000 for health care monitoring in accordance with various aspects of the present disclosure. For clarity, the method 1000 is described below with reference to aspects of one or more of the systems 100, 500, 600, 700, health care devices 110, and monitoring modules 210 described above with reference to FIGS. 1-8.

At block 1005, the method 1000 may include incorporating at least one function of the defined functions associated with a health care monitoring process into at least one health care device of a number of health care devices. The functions may be defined by, for example, the equipment providers, the health care providers, a standards or other industry group or association, and so forth. The functions may be incorporated into the health care device(s) by including software and/or hardware monitoring modules within the respective health care devices, either at manufacture or during use, by the equipment provider or by a user of the health care devices.

At block 1010, the method 1000 may include utilizing the at least one health care device to perform a subset of the defined functions associated with the health care functions associated with the health care monitoring process. The at least one health care device may be utilized by, for example, a health care provider, a patient himself or herself, and so forth.

It should be noted that the method 1000 is just one implementation and that the operations of the method 1000 may be rearranged or otherwise modified such that other implementations are possible.

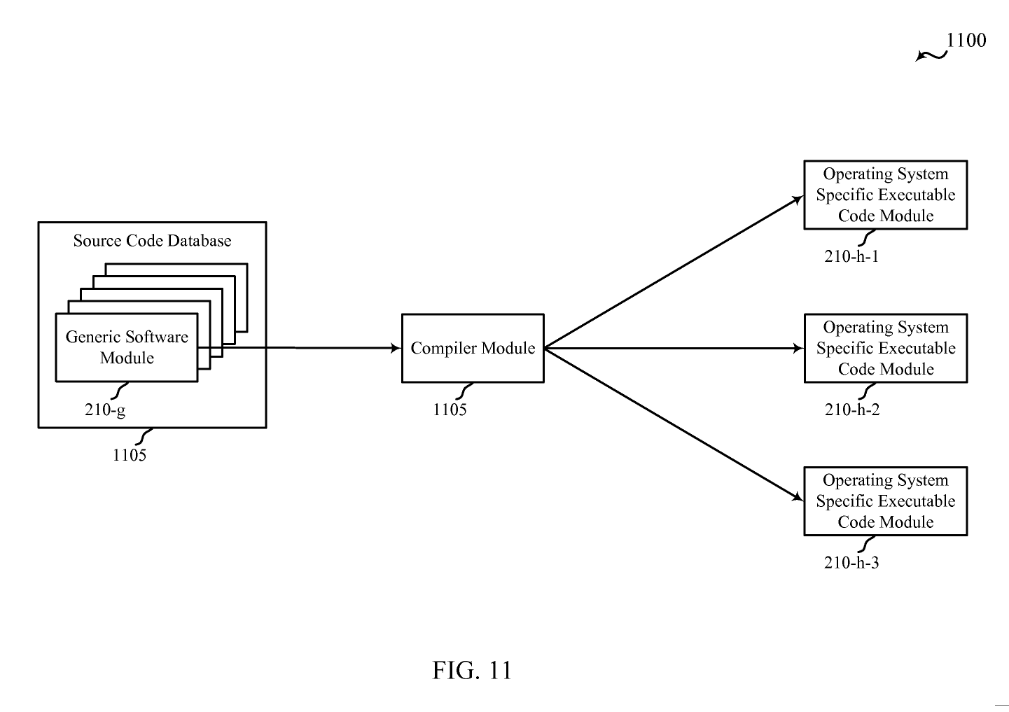

FIG. 11 is a block diagram of a multi-platform health care monitoring system 1100 that may be used in conjunction with one or more of the systems 100, 500, 600, 700 described above with reference to FIGS. 1-8—for example, the system 1100 shown in FIG. 11 may be used to generate one or more of the monitoring modules 210 shown and described with reference to FIG. 2A. As such, the system 1100 shown in FIG. 11 may carry out block 910 and/or block 1005 in methods 900, 1000 described above with reference to FIGS. 9 and 10.

The system 1100 shown in FIG. 11 includes a compiler module 1105 that may be configured to compile a generic software modules 210-g from a source code database 1110 into executable code modules 210-h-1, 210-h-2, 210-h-3 corresponding to a number of different operating systems. The source code database 1110 may include a number of generic software modules 210-g, with each of the generic software modules 210-g configured to implement a respective function associated with a patient monitoring process (which may be defined, e.g., as described above with reference to FIG. 1). The compiler module 1105 may be a commercial software program configured to compile each generic software module 210-g into operating system specific executable code modules 210-h-1, 210-h-2, 210-h-3 for use with different operating systems. The different operating systems may be associated with different health care monitoring devices—for example, different smartphones and tablet computers may have different operating systems, and the compiler module 1105 may produce code that can be executed by those different operating systems. In some embodiments, the different health care monitoring devices may be from different manufacturers—for example, a display device may be from a different manufacturer than a sensor device. However, the executable code modules 210-h-1, 210-h-2, 210-h-3 may be configured to publish data to a common data bus in a common data format regardless of the specific operating system corresponding to the executable code modules 210-h-1, 210-h-2, 210-h-3. In this manner, health care devices from many different manufacturers and of many different types may cooperate together in accomplishing the some or all of the functions of the patient monitoring process.

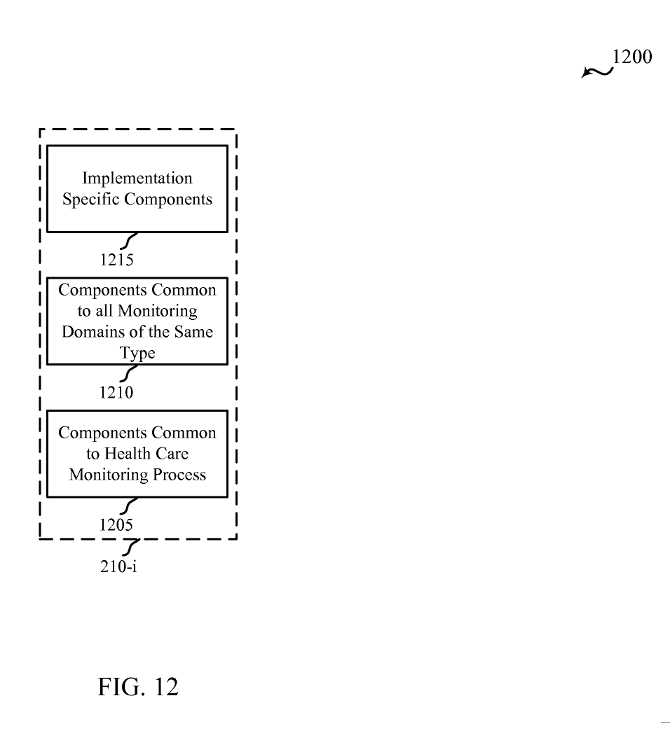

FIG. 12 is a block diagram 1200 of an operating system specific executable code module 210-i that may be generated by the system 1100 shown in FIG. 11 and that may be used as one of the monitoring modules 210 described above with reference to FIG. 2A. As shown in FIG. 12, the operating system specific executable code module 210-i includes components 1205 common to the entire, overarching health care monitoring process, components 1210 common to all monitoring domain (modules) of the same type as the executable code module 210-i, and implementation specific components 1215 to the executable code module 210-i itself.

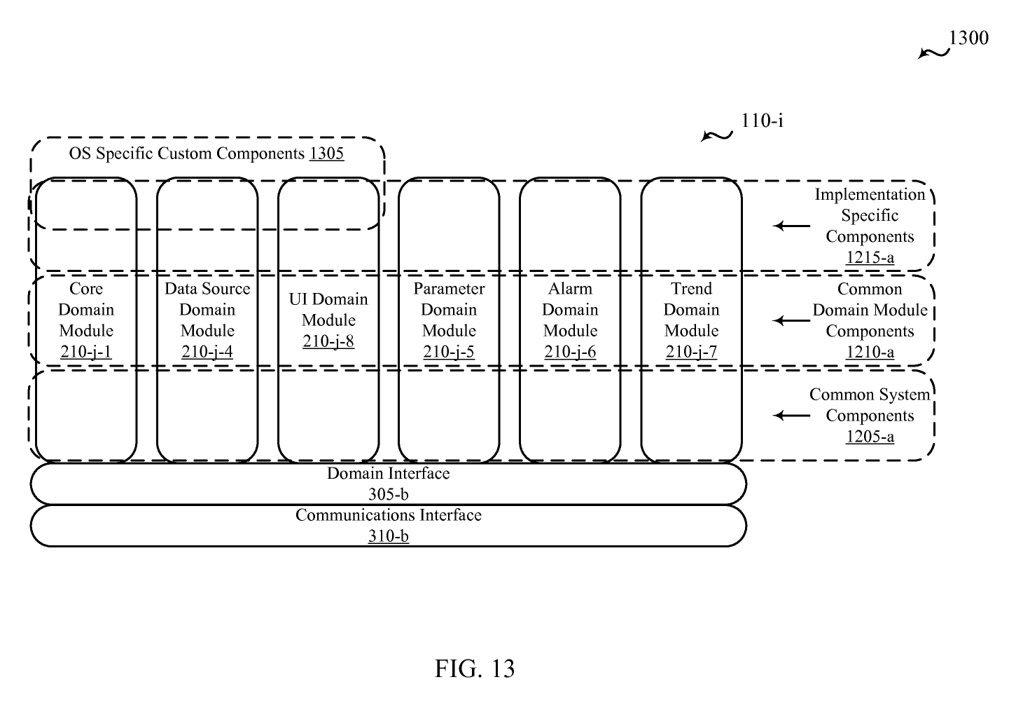

The components 1205 common to the entire, overarching health care monitoring process may include software and/or hardware needed to comply with communications and data format protocols, foundational software/hardware objects, and so forth. The components 1210 common to all monitoring domain (modules) of the same type may include software and/or hardware needed to implement a specific monitoring function. As one example, the components 1210 common to all monitoring domains of the same type for the alarm domain module 210-j-6 may include software and/or hardware needed to compare various parameters with preset thresholds and software and/or hardware needed to generate alarms. As another example, the components 1210 common to all monitoring domains of the same type for the parameter domain module 210-j-5 may include software and/or hardware needed to receive and interpret raw measurements from the data source domain module 210-j-4 and to generate parameters corresponding thereto. The implementation specific components 1215 may include software and/or hardware specific to the instantiation of the monitoring module. For example, for a core domain monitoring module, a data source domain monitoring module, and a user interface domain monitoring module, the implementation specific components 1215 may include operating system specific custom components (see box 1305 in FIG. 13). As another example, the implementation specific components 1215 of parameter and alarm domain monitoring modules may include software and/or hardware for receiving specific types of data and parameters and specific thresholds for the alarms.

Referring now to FIG. 13 and also still to FIG. 12, FIG. 13 shows a block diagram 1300 of a health care device 110-i with a number of specific implementations of monitoring modules 210-j-1, 210-j-4, 210-j-8, 210-j-2, 210-j-5, 210-j-6, 210-j-7, each of which has some common system components 1205-a (like the components 1205 common to the health care monitoring process in FIG. 12), some common domain module components 1210-a (like the components 1210 common to all monitoring domains of the same type in FIG. 12), and some implementation specific components 1215-a (like the implementation specific components 1215 in FIG. 12), including operating system specific custom components 1305 for at least some of the monitoring modules 210-j-1, 210-j-4, 210-j-8. The health care device 110-i in FIG. 13 also includes a domain interface 305-b and a communications interface 310-b, which may be examples of one or more aspects of the domain interfaces 305 and communications interfaces 310 shown and described above with reference to FIGS. 3 and 4.

FIG. 13 illustrates that when the system 1100 in FIG. 11 is used to generate monitoring modules, in some embodiments, substantial amounts of common software code can be re-used, which may reduce development and testing for new software components. Referring to FIGS. 11-13, it can also be appreciated that standardizing and modularizing the various functions associated with a health care monitoring process may allow flexibility to use one or many health care devices that are similar or different from one another, including being from different manufacturers. However, even if many health care devices of different types, different manufacturers, different purpose, and so forth, are used, they may nonetheless be interoperable in the health care monitoring process if they each publish and subscribe to data on a common data bus using a common data format because each health care device may be agnostic as to both the source of received data and the destination for published data.

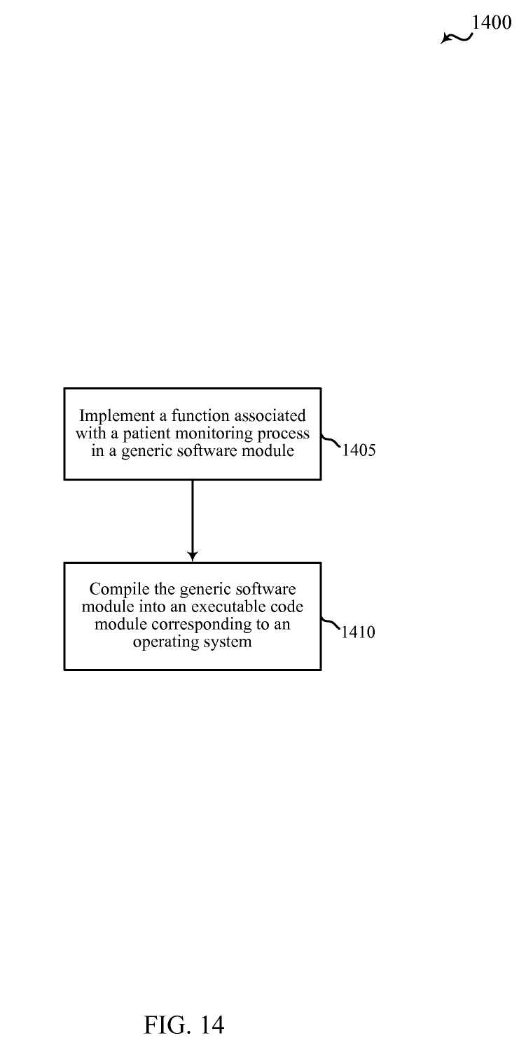

FIG. 14 is a flow chart illustrating an example of a method 1400 for compiling operating system specific monitoring modules, in accordance with various aspects of the present disclosure. For clarity, the method 1400 is described below with reference to aspects of one or more of the systems 100, 500, 600, 700, 1100 described above with reference to FIGS. 1 through 13.

At block 1405, the method 1400 may include implementing a function associated with a patient monitoring process in a generic software module. The generic software module may be generated by, for example, an equipment provider and/or may be defined by an industry standard.

At block 1410, the method 1400 may include compiling the generic software module into an executable code module corresponding to a specific operating system. The generic software module may be compiled by, for example, the compiler module 1105 in FIG. 11. Referring to the block diagram 1200 in FIG. 12, the generic software module may, before being compiled at block 1410, include the components 1205 common to the health care monitoring process, the components 1210 common to all monitoring domains of the same type, and, in some embodiments, at least some implementation specific components 1215 (e.g., those that are not operating system specific, but are specific to that monitoring module). After being compiled from the generic software module into an executable code module at block 1410, the executable code module may be used as a monitoring module specific to a certain health care device in one of the systems 100, 500, 600, 700 described above with reference to FIGS. 1 through 10.

It should be noted that the method 1400 is just one implementation and that the operations of the method 1400 may be rearranged or otherwise modified such that other implementations are possible.

FIG. 15 is a block diagram of a system 1500 for managing one or more health care devices, such as the health care devices 110 described above with reference to FIGS. 1 through 14. The system 1500 in FIG. 15 includes a remote device management server 1505 and/or a remote device management tool 1510 configured to send instructions to and/or receive information from one or more health care devices 110-j of the health care monitoring data network 105-d. The health care devices 110-j and network 105-d may be examples of one or more aspects of the health care devices 110 and networks 105 shown and described above with reference to FIGS. 1 through 14.

The remote device management server 1505 and/or the remote device management tool 1510 may be implemented with one or more application-specific integrated circuits (ASICs) adapted to perform some or all of the applicable functions in hardware. Alternatively, the functions may be performed by one or more other processing units (or cores), on one or more integrated circuits. In other embodiments, other types of integrated circuits may be used (e.g., Structured/Platform ASICs, Field Programmable Gate Arrays (FPGAs), and other Semi-Custom ICs), which may be programmed in any manner known in the art. The functions of the remote device management server 1505 and/or the remote device management tool 1510 may also be implemented, in whole or in part, with instructions embodied in a memory (e.g., as a software module), formatted to be executed by one or more general or application-specific processors. In some embodiments, the remote device management server 1505 may include one or more aspects of the server 115-d shown in FIG. 8.

The remote device management server 1505 and/or the remote device management tool 1510 may be configured to couple with one or more of the health care devices 110-j over a network, to send instructions to the one or more health care devices 110-j relating to their use, and receive information back from the one or more health care devices 110-j responsive to the instructions. In some embodiments, the remote device management server 1505 may be located on the same premises as the health care devices 110-j (i.e., within the same health care facility) even if not located in, for example, the same room. In these embodiments, the remote device management server 1505 is “remote” in that it is in a centralized location relative to the health care devices 110-j. In other embodiments, however, the remote device management server 1505 may truly be located remotely from the health care devices 110-j (e.g., in a different city or a different part of the same city). In either case, the health care devices 110-j may be coupled with a network (e.g., network 105-d in FIG. 15) in order to communicate with the remote device management server 1505 and/or the remote device management tool 1510. The network coupling the health care devices 110-j with the remote device management server 1505 and/or the remote device management tool 1510 may be a wireless local area network (WLAN), a wireless wide area network (WWAN), a wired local area network (LAN) such as Ethernet, and so forth. The remote device management tool 1510 may be used in some but not all embodiments either as a standalone remote device management device, or as an intermediary to the remote device management server 1505.

The remote devices management tool 1510 may be used as an intermediary to the remote device management server 1505 in at least two ways. As one example, the remote device management tool 1510 may establish a connection between the health care device 110-j and the remote device management server 1505. As another example, the remote device management tool may receive instructions from the remote device management server 1505, carry out those instructions, and then reconcile with the remote device management server after completing the instructions. The reconciliation may include informing the remote device management server 1505 which operations were carried out and also may include transmitting to the server 1505 any data that was received from the health care devices 110-j.

Referring still to FIG. 15, in some embodiments, the remote device management server 1505, the remote device management tool 1510, the network 105-d, and/or the health care devices 110-j themselves may be configured to determine relative costs associated with a number of different network technologies for communications between the health care devices 110-j and the remote device management server 1505 and/or remote device management tool 1510, or for communications between the remote device management tool 1510 and the remote device management server 1505. As just one example, the relative costs of using a health care facility Wi-Fi connection versus a cellular network data connection may be determined, and these determined relative costs may be utilized to select one of the different network technologies for communications.

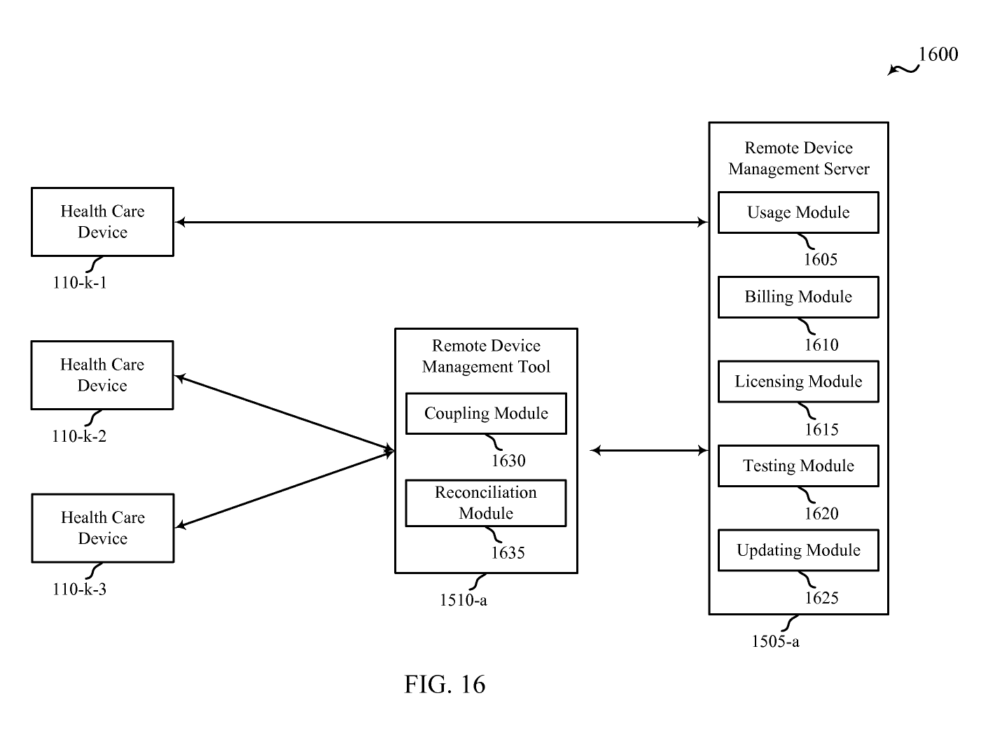

FIG. 16 is a block diagram of a system 1600 for managing one or more health care devices, such as the health care devices 110 described above with reference to FIGS. 1 through 15. The system 1600 in FIG. 16 may be an example of aspects of the system 1500 in FIG. 15, and includes a remote device management server 1505-a and a remote device management tool 1510-a, which may be examples of aspects of the remote device management server 1505 and the remote device management tool 1510 in FIG. 15.

The remote device management server 1505-a and/or the remote device management tool 1510-a (including their respective modules 1605, 1610, 1615, 1620, 1625, 1630, 1635, which will be described below) may be implemented with one or more application-specific integrated circuits (ASICs) adapted to perform some or all of the applicable functions in hardware. Alternatively, the functions may be performed by one or more other processing units (or cores), on one or more integrated circuits. In other embodiments, other types of integrated circuits may be used (e.g., Structured/Platform ASICs, Field Programmable Gate Arrays (FPGAs), and other Semi-Custom ICs), which may be programmed in any manner known in the art. As another alternative, the functions may be implemented, in whole or in part, with instructions embodied in a memory (e.g., as a software module), formatted to be executed by one or more general or application-specific processors.

In the example shown in FIG. 16, a first health care device 110-k-1 is shown directly coupled with the remote device management server 1505-a, whereas other health care devices 110-k-2, 110-k-3 are shown directly coupled with the remote device management tool 1510-a, which serves as an intermediary to the remote device management server 1505-a. In general, however, any health care device 110 may be directly and/or indirectly coupled with either the remote device management server 1505-a and/or the remote device management tool 1510-a.

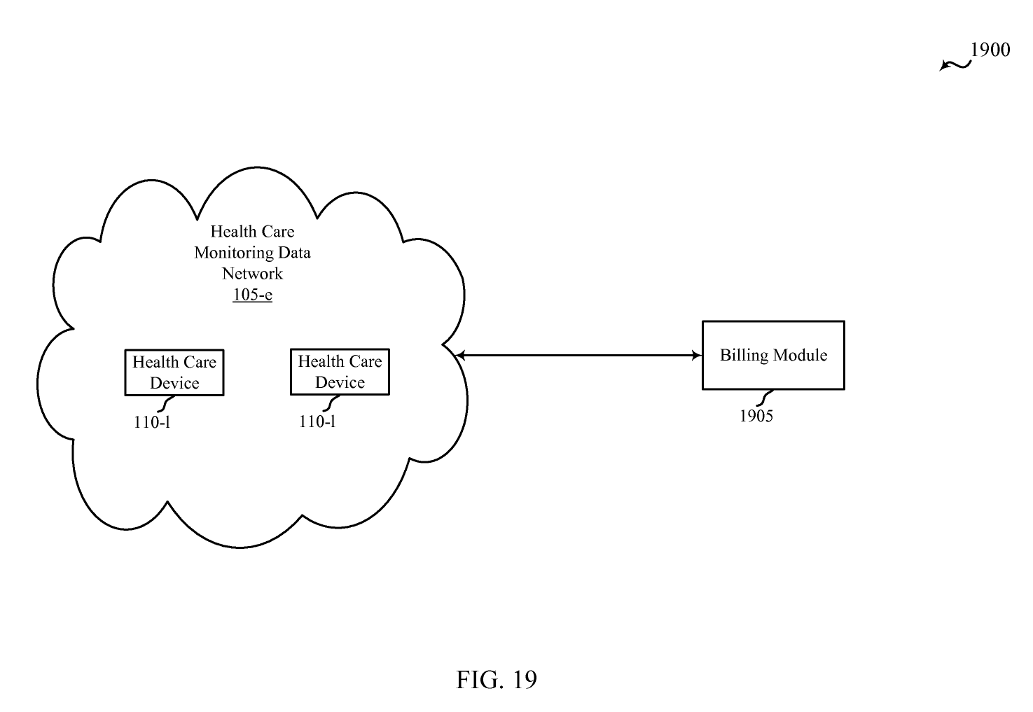

The remote device management server 1505-a in FIG. 16 include several modules 1605, 1610, 1615, 1620, 1625 that may implement certain respective device management functions. For example, the usage module 1605 may be configured to request usage information from the health care devices 110-k-1, 110-k-2, 110-k-3, and to interpret returned usage information (which may be in the form of a usage log). The usage information may be used by the remote device management server 1505-a for many different purposes, including to generate billing information in the billing module 1610 based on specific uses of the health care devices 110-k-1, 110-k-2, 110-k-3, which will also be described below with reference to FIGS. 19-21.

The licensing module may be configured to manage licenses related to the health care devices 110-k-1, 110-k-2, 110-k-3, including administering and verifying license keys. In one example, the licensing module may transmit licensing information to a health care device 110-k-1, 110-k-2, 110-k-3 after a license is purchased by the equipment purchaser. The licensing information may activate some or all of the functions of the health care device 110-k-1, 110-k-2, 110-k-3. In some instances, the licensing information provided to the health care devices 110-k-1, 110-k-2, 110-k-3 by the licensing module may include activation instructions for demoing an unlicensed function that the health care device 110-k-1, 110-k-2, 110-k-3 is capable of performing but that the user has not yet purchased. Because these kinds of licensing information can be sent to the health care devices 110-k-1, 110-k-2, 110-k-3 from the remote device management server 1505-a, in some embodiments, no person may need to physical go to the location of the health care devices 110-k-1, 110-k-2, 110-k-3, or, if they do need to go to the physical location, the licensing can be done quickly and wirelessly (e.g., using the remote device management tool 1510-a as an intermediary).

The testing module 1620 may be configured to order certain self-tests by done by the health care devices 110-k-1, 110-k-2, 110-k-3 in order to check status and operability, and may further be configured to interpret and act upon the results of the self-tests.

The updating module 1625 may be configured to determine whether any software/firmware/hardware updates are available for each health care device 110-k-1, 110-k-2, 110-k-3, and, in some embodiments, to push certain software/firmware updates to the health care devices 110-k-1, 110-k-2, 110-k-3 and/or create service tickets for hardware updates to be physically installed. The updating module 1625 may also be configured to alter the device configuration for each of the health care devices 110-k-1, 110-k-2, 110-k-3 as needed.

The remote device management tool 1510-a also includes several modules 1630, 1635 that may implement certain respective device management functions. The coupling module 1630, for example, may be configured to couple with the health care devices 110-k-1, 110-k-2, 110-k-3, and, in some embodiments, to couple the health care devices 110-k-1, 110-k-2, 110-k-3 with the remote device management server 1505-a. The coupling with the health care devices 110-k-1, 110-k-2, 110-k-3 and/or the coupling of the health care devices 110-k-1, 110-k-2, 110-k-3 with the remote device management server 1505-a may be automatically performed by the coupling module 1630 if the health care devices 110-k-1, 110-k-2, 110-k-3 are detected on a wireless network associated with the remote device management tool 1510-a in some embodiments. Further, certain other functions (e.g., automatically updating the heath care devices 110-k-1, 110-k-2, 110-k-3) may also be automatically performed once the health care devices 110-k-1, 110-k-2, 110-k-3 are coupled with the network via coupling module 1630.

As mentioned above, certain health care devices 110-k-1, 110-k-2, 110-k-3 may include a local database configured to store data (e.g., in the processing module and storage 215 of FIG. 2A), whereas other health care devices 110-k-1, 110-k-2, 110-k-3 do not have a local database for storing monitoring data. For both types of health care devices 110-k-1, 110-k-2, 110-k-3, though, the system 1600 shown in FIG. 16 may be used to transmit stored monitoring information to the remote device management server 1505-a-which may store the received data itself or may forward it to other servers, such as the server 115 in the system 100 shown in FIG. 1. For those health care devices 110-k-1, 110-k-2, 110-k-3 that do include a local database configured to store monitoring data, the coupling module 1630 may retrieve and transmit stored data from the local database to the remote device management server 1505-a, whereas for those health care devices 110-k-1, 110-k-2, 110-k-3 that do not include a local database configured to store monitoring data, the coupling module 1630 may transmit data generated by the health care devices 110-k-1, 110-k-2, 110-k-3 to the remote device management server 1505-a in substantially real-time as the data is generated.

The reconciliation module 1635 of the remote device management tool 1510-a may be configured to allow the remote device management tool 1510-a to be used in an “offline” mode to manage the health care devices 110-k-1, 110-k-2, 110-k-3, and to then report back to the remote device management server 1505-a once a network connection to the remote device management server 1505-a is once again available for use.

Referring still to FIG. 16, it should be noted that while a certain configuration of the remote device management server 1505-a and remote device management tool 1510-a are shown, other configurations are possible, including one in which the entirety of the modules and functionality described with reference to the remote device management server 1505-a is included within the remote device management tool 1510-a, thereby obviating the need for a separate remote device management server. As another example, no remote device management tool may be used, and the health care devices 110-k-1, 110-k-2, 110-k-3 may instead be coupled directly with the remote device management server 1505-a. Many other configurations are also within the scope of the present disclosure. As another example, in some embodiments, the health care devices 110-k-1, 110-k-2, 110-k-3 may be self-contained (e.g., stand-alone) devices, but may still be accessible by a separate health care device or mobile application capable of interacting with the health care devices 110-k-1, 110-k-2, 110-k-3 (e.g., by interrogating the devices for data, issuing commands, providing updates, retrieving usage logs, providing licensing keys, and so forth. In this manner, while no centralized, remote device management server interacts with the health care devices 110-k-1, 110-k-2, 110-k-3, other health care devices can provide similar functionality. As still another example, in some embodiments, multiple health care devices 110 may be used to connect each other to the remote device management server 1505-a (e.g., in a device-to-device or node-to-node architecture). In these embodiments, the health care devices 110-k-1, 110-k-2, 110-k-3 may be configured to operate in either a connected or non-networked state (e.g., regardless of their connection or lack thereof to the remote device management server 1505-a), and may be configured to store and forward data as appropriate.

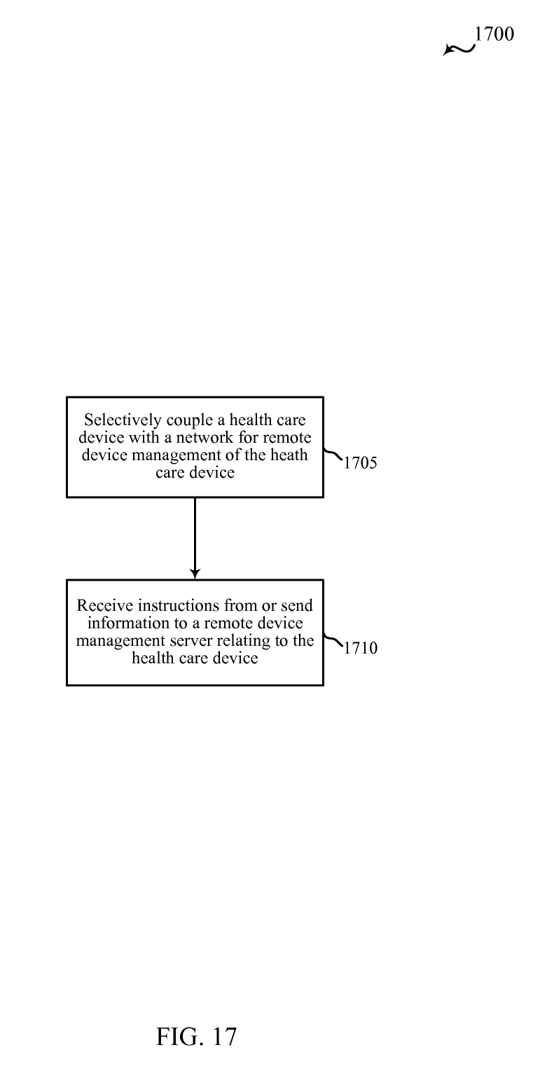

FIG. 17 is a flow chart illustrating an example of a method 1700 for managing one or more health care devices in accordance with various aspects of the present disclosure. For clarity, the method 1700 is described below with reference to aspects of one or more of the systems 1500, 1600 described with reference to FIGS. 15 and 16, and also with reference to aspects of one or more of the systems 100, 500, 600, 700, 1100 described above with reference to FIGS. 1 through 14. In some embodiments, the method 1700 may be implemented by a health care device, such as the health care devices 110 described above.

At block 1705, the method 1700 may include selectively coupling a health care device with a network for remote device management of the health care device. The selectively coupling of the health care device to the network may be performed by, for example, the health care device itself, the network itself, or through an intermediary such as the remote device management tool 1510 in FIG. 15.

At block 1710, the method 1700 may include receiving instructions from and/or sending information to a remote device management server relating to the health care device. In some examples, the health care device may receive an instruction from the remote device management server, and may send information responsive to the received instruction.

It should be noted that the method 1700 is just one implementation and that the operations of the method 1700 may be rearranged or otherwise modified such that other implementations are possible.

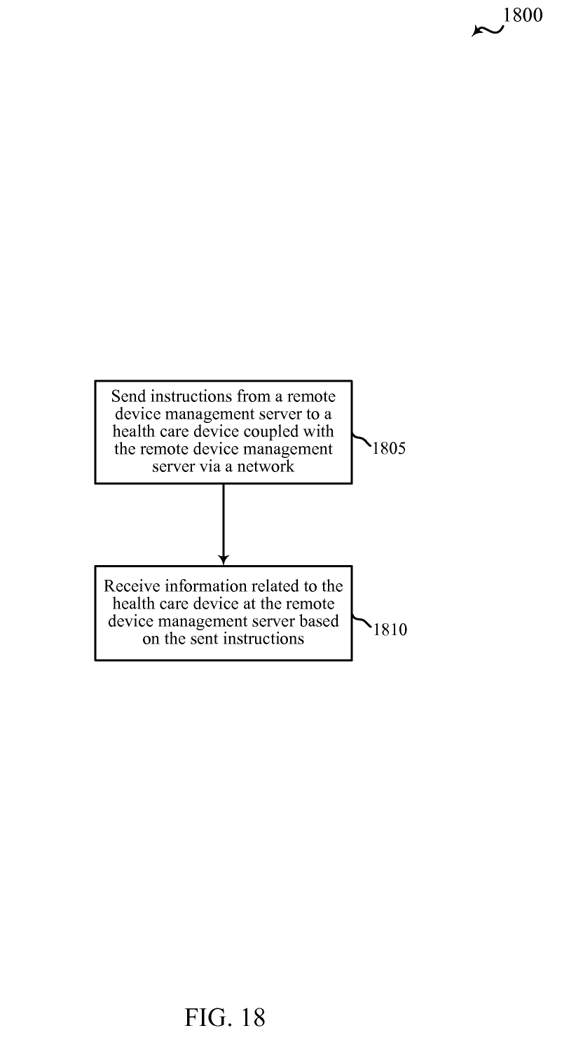

FIG. 18 is a flow chart illustrating an example of a method 1700 for managing one or more health care devices in accordance with various aspects of the present disclosure. For clarity, the method 1800 is described below with reference to aspects of one or more of the systems 1500, 1600 described with reference to FIGS. 15 and 16, and also with reference to aspects of one or more of the systems 100, 500, 600, 700, 1100 described above with reference to FIGS. 1 through 14. More specifically, the method 1800 may be implemented by a remote device management server or tool, such as the remote device management server 1505 or the remote device management tool 1510 in FIG. 15.

At block 1805, the method 1800 may include sending instructions from a remote device management server to a health care device coupled with the remote device management server via a network.

At block 1810, the method 1800 may include receiving information related to the health care device at the remote device management server based at least in part on the instructions sent at block 1805.

It should be noted that the method 1800 is just one implementation and that the operations of the method 1800 may be rearranged or otherwise modified such that other implementations are possible.

FIG. 19 is a block diagram of a system 1900 that may be used to charge for use of one or more health care devices, such as the health care devices 110 described above with reference to FIGS. 1 through 18. The system 1900 in FIG. 19 includes a billing module 1905 configured to determine charges and generate billing reports based on identifiable uses of one or more health care devices (including the hardware and/or software of each health care device). The health care devices 110-1 and network 105-e in FIG. 19 may be examples of one or more aspects of the health care devices 110 and networks 105 shown and described above with reference to FIGS. 1 through 18.

The billing module 1905 may be implemented with one or more application-specific integrated circuits (ASICs) adapted to perform some or all of the applicable functions in hardware. Alternatively, the functions may be performed by one or more other processing units (or cores), on one or more integrated circuits. In other embodiments, other types of integrated circuits may be used (e.g., Structured/Platform ASICs, Field Programmable Gate Arrays (FPGAs), and other Semi-Custom ICs), which may be programmed in any manner known in the art. The functions of the billing module 1905 may also be implemented, in whole or in part, with instructions embodied in a memory (e.g., as a software module), formatted to be executed by one or more general or application-specific processors. In some embodiments, the billing module 1905 may include one or more aspects of the server 115-d shown in FIG. 8. Also, in some embodiments, the billing module 1905 in FIG. 19 may include one or more aspects of the billing module 1610 in FIG. 16, and may be implemented within a remote device management server 1505 as described above with reference to FIGS. 15 and 16, or within a server 115 described with reference to the system 100 in FIG. 1. In some embodiments, the billing module 1905 may be positioned proximate the health care devices 110-1 (e.g., within the same health care facility), whereas in other embodiments the billing module 1905 may be in a remote location from the health care devices 110-1 (e.g., in a different city or a different part of the same city) but coupled therewith through a network connection.

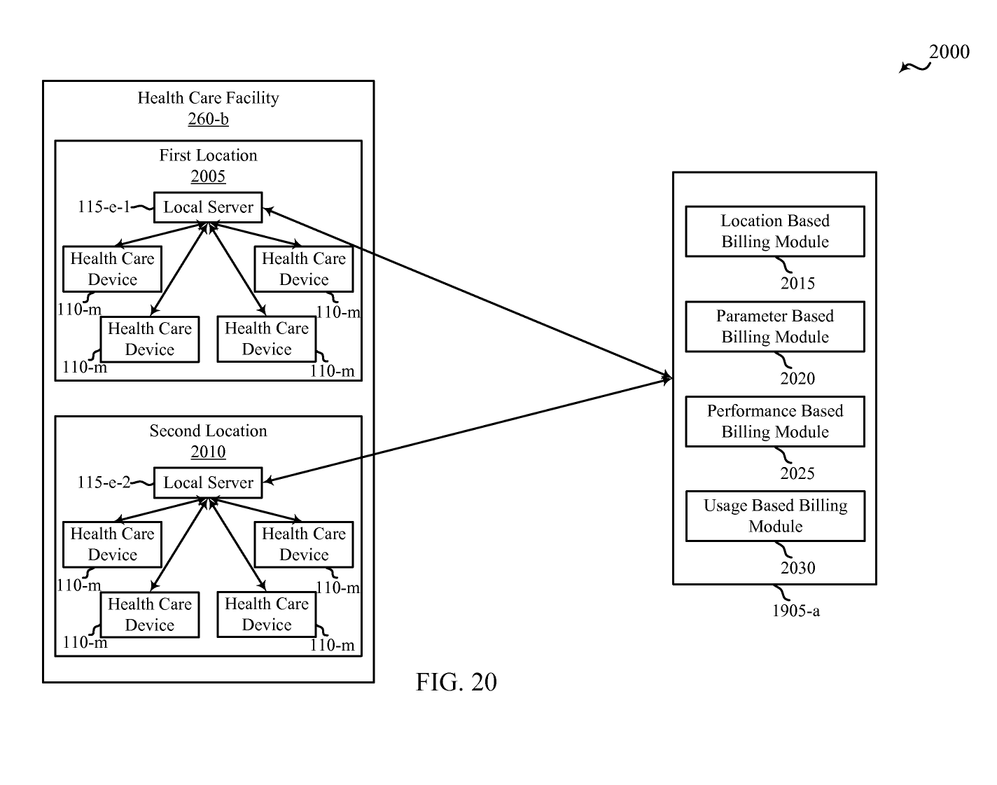

In operation, the health care devices 110-1 (or a server coupled therewith, or a health care provider, etc.) may track the usage of the health care devices 110-1, including, for example, how, where, when, and for what purpose each health care device 110-1 is used. The billing module 1905 may receive this information regarding one or more identifiable uses of a health care device and determine charges corresponding to each identifiable use of the health care device based on one or more billing methods described in more detail with reference to FIG. 20. The billing module 1905 may also generate billing reports based on the determined charges for each identifiable use of the health care devices, and transmit the billing reports to customers, such as the users of the relevant health care devices. In some embodiments, the usage of the health care device may be on a device level—e.g., if any portion of the device is used, a charge will be associated with use of the overall device. In other embodiments, usage of some portion of the health care device—such as a particular software module—may be tracked separately from usage of the overall device, and the billing module 1905 may base the billing reports on the specific, tracked usage and/or the overall device usage.

Turning now to FIG. 20, a block diagram of a system 2000 for managing one or more health care devices is shown, which may be an example of one or more aspects of the system 1900 in FIG. 19. The system 2000 in FIG. 20 includes a billing module 1905-a, with a location based billing module 2015, a parameter based billing module 2020, a performance based billing module 2025, and a usage based billing module 2030. The billing module 1905-a is coupled with local servers 115-e-1, 115-e-2 in respective first and second locations 2005, 2010 within a health care facility, with health care devices 110-m coupled with respective local servers 115-e-1, 115-e-2. The billing module 1905-a, the health care devices 110-m, and the servers 115-e-1, 115-e-2 may be examples of one or more aspects of the billing module 1905, health care devices 110, and servers 115 described above with reference to FIGS. 1 through 19.

The location based billing module 2015, the parameter based billing module 2020, the performance based billing module 2025, and the usage based billing module 2030 may be implemented with one or more application-specific integrated circuits (ASICs) adapted to perform some or all of the applicable functions in hardware. Alternatively, the functions may be performed by one or more other processing units (or cores), on one or more integrated circuits. In other embodiments, other types of integrated circuits may be used (e.g., Structured/Platform ASICs, Field Programmable Gate Arrays (FPGAs), and other Semi-Custom ICs), which may be programmed in any manner known in the art. As another alternative, the functions may be implemented, in whole or in part, with instructions embodied in a memory (e.g., as a software module), formatted to be executed by one or more general or application-specific processors.

The operation of each of the specific billing modules 2015, 2020, 2025, 2030 will be described shortly, but note that in some embodiments, the use of these billing modules may allow an equipment provider to supply one or more health care devices 110-m at no charge to users. Instead, the specific billing modules 2015, 2020, 2025, 2030 may be used to charge for the actual use of the health are devices 110-m, with the exact charge depending on which billing mechanism is used by the equipment provider.

The location based billing module 2015 may be configured to determine charges corresponding to identifiable uses of a health care device 110-m based on a location in which the health care device 110-m is used. For example, in a given health care facility 260-b, there may be at least two different locations 2005, 2010, and the location based billing module 2015 may associate different charges for use of similar or identical health care devices 110-m in different locations. Specifically, the charge per use of a health care device 110-m in the first location 2005 may be more or less than the charge per use of the same health care device 110-m in the second location 2010. In some embodiments, each health care device 110-m may include a positioning module to determine in which of the locations 2005, 2010 the device was used, and this information may be provided to the location based billing module 2015. In other embodiments, the location information may be manually entered by a health care provider, or may be inferred by the local server 115-e-1, 115-e-2 through which the usage information is transmitted to the billing module 1905-a.

The parameter based billing module 2020 may be configured to determine charges corresponding to identifiable uses of a health care device 110-m based on which parameters the device was used to monitor. For example, there may be many different health care devices 110-m that can take a single physiological measurement. Instead of charging users for use of a specific health care device 110-m (regardless of how the health care device 110-m is used), the parameter based billing module 2020 may receive information relating to how the health care device 110-m was used (e.g., which parameter(s) was/were measured), and may associate similar charges with similar uses, regardless of the equipment used. In this manner, the cost to measure, for example, a patient's heart rate may be the same regardless of whether a simple electrode or a complex machine is used to monitor the heart rate.

The performance based billing module 2025 may be configured to determine charges corresponding to identifiable uses of a health care device 110-m based on an outcome of using the health care device in a patient monitoring process, such as whether there is an improvement in the delivery of health care services. For example, the manufacturer of a health care device 110-m may determine, using the performance based billing module 2025, the charge corresponding to the use of the health care device 110-m based on whether the health care device 110-m performed correctly. If the health care device 110-m malfunctioned or was not able to measure a certain physiological parameter, for example, then there would be no costs associated with that use. By the same token, if a particular health care device 110-m is shown to improve how health care services are delivered, or shown to improve the health of patients, then there may be a charge associated with that use. For example, if a health care facility is contemplating using tablet computers for its staff, but is unsure whether the investment typically required to purchase the tablet computers would be worthwhile, an equipment provider may supply the tablet computers at no charge to the health care facility, monitor the productivity of the health care providers, and, if the productivity does increase, then the equipment provider may charge for the use of the tablet computers. On the other hand, if there is no change in productivity of the health care workers, or the productivity worsens, then the equipment provider may not charge for the use of the tablet computers. Generally speaking, the performance based billing module 2025 may incorporate a wide variety of performance based rules to determine when, and how much, each identifiable use of a health care device 110-m will be charged to the user.

The usage based billing module 2030 may be configured to determine charges corresponding to identifiable uses of a health care device 110-m based on which portions of the hardware and/or software of the health care device 110-m were used to monitor a patient. For example, the health care device 110-m may include several different sensors, several different software modules, and so forth. If usage of these various components of the health care device 110-m are tracked and provided to the usage based billing module 2030, the charge for using the health care device 110-m may be based at least in part on this usage information. In some embodiments, the overall use of the device may also be tracked, and the overall charge for using the health care device may be based on either usage of the overall device, usage of the individual software and/or hardware components of the device, or a combination thereof.

While FIG. 20 illustrates several different types of billing modules 2015, 2020, 2025, 2030 that may be used, it will be appreciated that other types of billing may also be implemented in the system 2000 illustrated in FIG. 20. As just one example, a time based billing module may determine charges for using the health care devices 110-m based on the times at which the health care devices 110-m are used.

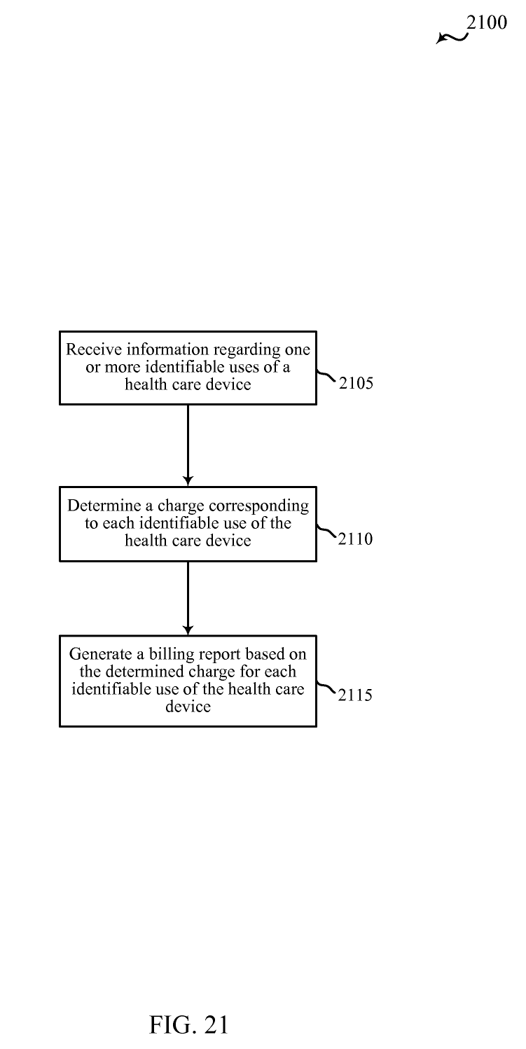

FIG. 21 is a flow chart illustrating an example of a method 2100 for charging for use of one or more health care devices, in accordance with various aspects of the present disclosure. For clarity, the method 2100 is described below with reference to aspects of one or more of the systems 1900, 2000 described with reference to FIGS. 19 and 20, and also with reference to aspects of one or more of the systems 100, 500, 600, 700, 1100, 1500, 1600 described above with reference to FIGS. 1 through 18. In some embodiments, the method 2100 may be implemented by a billing module 1905, 1905-a, 2015, 2020, 2025, 2030.

At block 2105, the method 2100 may include receiving information regarding one or more identifiable uses of a health care device. At block 2110, the method 2100 may include determining a charge corresponding to each identifiable use of the health care device. And at block 2115, the method 2100 may optionally include generating a billing report based on the determined charge for each of the identifiable uses of the health care device.

It should be noted that the method 2100 is just one implementation and that the operations of the method 2100 may be rearranged or otherwise modified such that other implementations are possible.

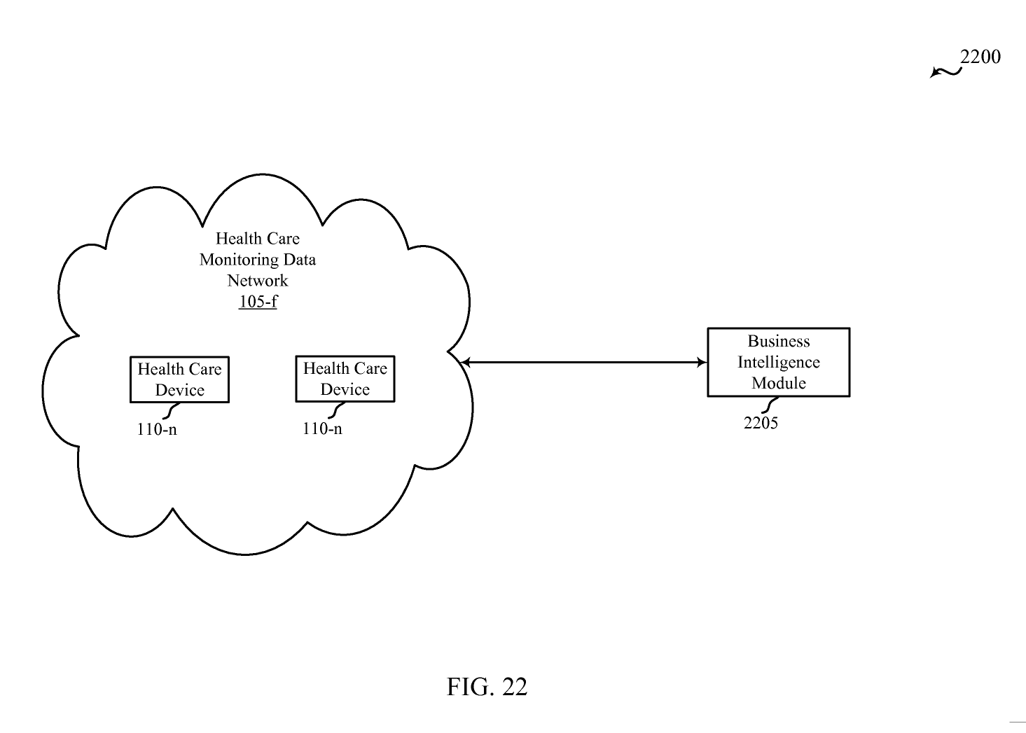

FIG. 22 is a block diagram of a system 2200 that may be used to analyze data from one or more health care devices, such as the health care devices 110 described above with reference to FIGS. 1 through 21. The system 2200 in FIG. 22 includes a business intelligence module 2205 configured to generate business intelligence based on data received from the health care devices 110-n. The data may be sent via network 105-f over a wireless or wired network connection. The health care devices 110-n and network 105-f in FIG. 22 may be examples of one or more aspects of the health care devices 110 and networks 105 shown and described above with reference to FIGS. 1 through 21.

The business intelligence module 2205 may be implemented with one or more application-specific integrated circuits (ASICs) adapted to perform some or all of the applicable functions in hardware. Alternatively, the functions may be performed by one or more other processing units (or cores), on one or more integrated circuits. In other embodiments, other types of integrated circuits may be used (e.g., Structured/Platform ASICs, Field Programmable Gate Arrays (FPGAs), and other Semi-Custom ICs), which may be programmed in any manner known in the art. The functions of the business intelligence module 2205 may also be implemented, in whole or in part, with instructions embodied in a memory (e.g., as a software module), formatted to be executed by one or more general or application-specific processors. In some embodiments, the business intelligence module 2205 may include one or more aspects of the server 115-d shown in FIG. 8. Also, in some embodiments, the business intelligence module 2205 in FIG. 22 may be implemented within a remote device management server 1505 as described above with reference to FIGS. 15 and 16, or within a server 115 described with reference to the system 100 in FIG. 1. In some embodiments, the business intelligence module 2205 may be positioned proximate the health care devices 110-n (e.g., within the same health care facility), whereas in other embodiments the business intelligence module 2205 may be in a remote location from the health care devices 110-n (e.g., in a different city or a different part of the same city) but coupled therewith through a network connection.

In operation, the business intelligence module 2205 may receive data from one or more health care devices 110-n, and process the received data to generate business intelligence based on the received data. The data may be processed at a remote location from the health care devices 110-n, as mentioned above. The business intelligence module 2205 may also transmit the generated business intelligence to one or more locations. For example, the business intelligence module 2205 may transmit the generated business intelligence to a user of each health care device 110-n, to a server for receive by an administrator of the heath care device 110-n, and so forth.

Still referring to FIG. 22, in some embodiments, the business intelligence module 2205 may continuously receive data (e.g., in real-time) from the health care devices 110-n, whereas in other embodiments, the health care devices 110-n (or an intermediary device or server) may buffer the data and sent the buffered data to the business intelligence module only at predefined times (e.g., in post-time). In other embodiments, the health care devices 110-n only send relevant data to the business intelligence module 2205 when requested to do so by the business intelligence module 2205, or when a user of the health care devices 110-n requests that the data be sent to the business intelligence module 2205 (e.g., when there is an error, when the user needs assistance, etc.). In still other embodiments, the health care devices 110-n (or an intermediary device or server) may summarize the data from the health care devices 110-n and send only the summary of the data that otherwise would be available from the health care devices 110-n.

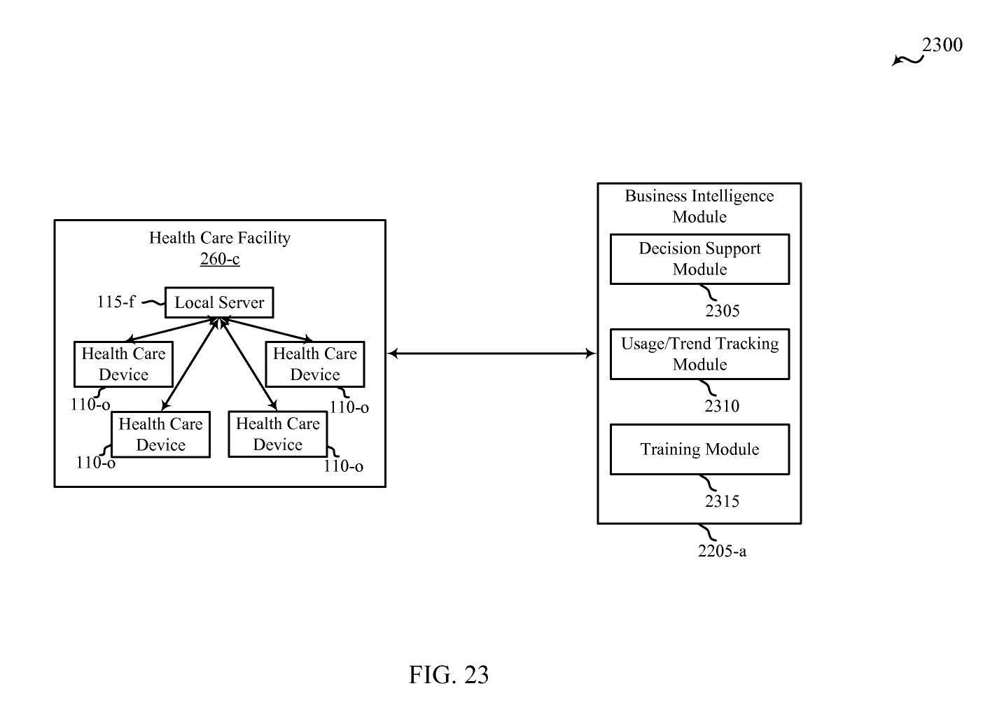

Turning now to FIG. 23, a block diagram of a system 2300 that may be used to analyze data from one or more health care devices is shown, which may be an example of one or more aspects of the system 2200 in FIG. 22. The system 2300 in FIG. 23 includes a business intelligence module 2205-a, with a decision support module 2305, a usage/trend tracking module 2310, and a training module 2315. The business intelligence module 2205-a is coupled with the health care facility 260-c, with health care devices 110-m coupled with a local servers 115-f at the health care facility 260-c. The business intelligence module 2205-a, the health care devices 110-o, and the servers 115-f may be examples of one or more aspects of the business intelligence module 2205, health care devices 110, and servers 115 described above with reference to FIGS. 1 through 22.

The decision support module 2305, the usage/trend tracking module 2310, and the training module 2315 may be implemented with one or more application-specific integrated circuits (ASICs) adapted to perform some or all of the applicable functions in hardware. Alternatively, the functions may be performed by one or more other processing units (or cores), on one or more integrated circuits. In other embodiments, other types of integrated circuits may be used (e.g., Structured/Platform ASICs, Field Programmable Gate Arrays (FPGAs), and other Semi-Custom ICs), which may be programmed in any manner known in the art. As another alternative, the functions may be implemented, in whole or in part, with instructions embodied in a memory (e.g., as a software module), formatted to be executed by one or more general or application-specific processors.

The decision support module 2305 may be configured to receive one or more parameters monitored by the health care devices 110-o and to generate and transmit decision support business intelligence to a health care provider using the health care devices 110-o. The usage/trend tracking module 2310 may be configured to receive usage information from the health care devices 110-o and generate and transmit trend business intelligence information to, for example, an administrator over the health care devices 110-o. In some embodiments, the usage/trend tracking module 2310 may also utilize predictive analysis for system assessment, may send preventative messaging related to needed interventions, and so forth. For example, the usage/trend tracking module 2310 may receive usage data from health care devices 110-o, and use that usage data to predict how many and which kinds of health care devices will be needed in the future, in order to allow a hospital or other health care facility to adjust their health care device inventory to match the predicted usage. Similarly, the usage/trend tracking module 2310 may receive health care data regarding a patient and provide messages related to interventions for the patient based on the received health care data.

The training module 2315 may be configured to receive usage information from the health care devices 110-o, and may generate and transmit training opportunities business intelligence to, for example, an administrator over the health care devices 110-o. The training opportunities business intelligence may identify one or more areas in which, based on the received usage information, one or more health care providers may be incorrectly using the health care devices 110-o, or may not be using the health care devices 110-o to their full potential. In other embodiments, the received usage information may be analyzed by the training module 2315 to identify best practices for a group or across groups of health care providers. In still other embodiments, the received usage information may be analyzed by an equipment provider to update/improve the health care devices, or to provide a more customized solution to the users of the health care devices.

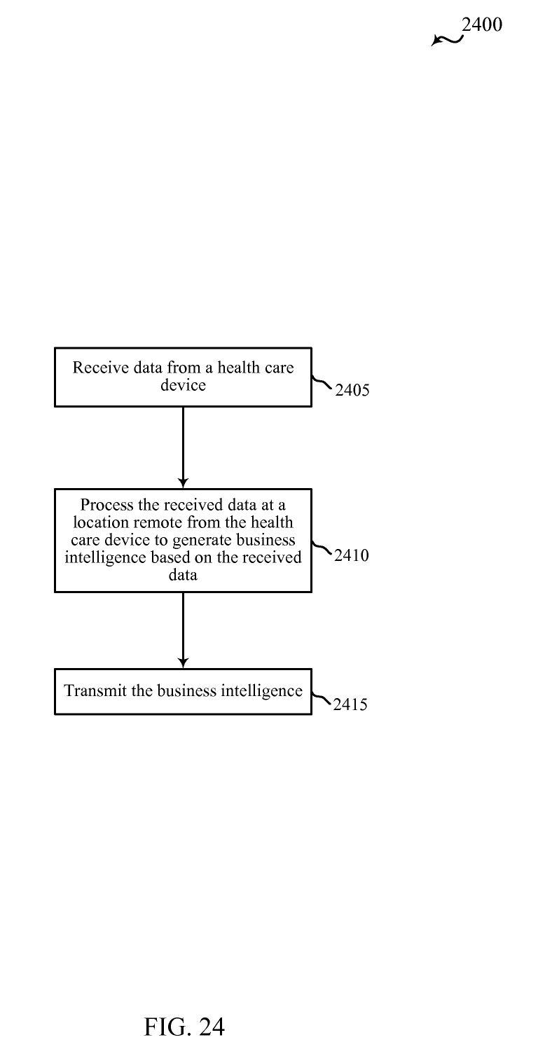

FIG. 24 is a flow chart illustrating an example of a method 2400 for analyzing data from one or more health care devices, in accordance with various aspects of the present disclosure. For clarity, the method 2400 is described below with reference to aspects of one or more of the systems 2200, 2300 described with reference to FIGS. 22 and 23, and also with reference to aspects of one or more of the systems 100, 500, 600, 700, 1100, 1500, 1600 described above with reference to FIGS. 1 through 18. In some embodiments, the method 2400 may be implemented by a business intelligence module 2205, 2205-a.

At block 2405, the method 2400 may include receiving data from a health care device over a network at a business intelligence module. At block 2410, the method 2400 may include processing the received data (optionally at a location remote from the health care device) to generate business intelligence based on the received data. The “remote” location may be, in some embodiments, a central location within a health care facility, for example, or may be in a different city or different part of the same city. At block 2415, the method 2400 may include transmitting the business intelligence to, for example, a user or administrator of the health care device.

It should be noted that the method 2400 is just one implementation and that the operations of the method 2400 may be rearranged or otherwise modified such that other implementations are possible.

While various embodiments have been described above, it should be understood that they have been presented by way of example only, and not limitation. Although various embodiments have been described as having particular features and/or combinations of components, other embodiments are possible having a combination of any features and/or components from any of embodiments as discussed above.

Where block diagrams and/or embodiments described above indicate certain components arranged in certain orientations or positions, the arrangement of components may be modified. While the embodiments have been particularly shown and described, it will be understood that various changes in form and details may be made.

The above description provides examples, and is not limiting of the scope, applicability, or configuration set forth in the claims. Changes may be made in the function and arrangement of elements discussed without departing from the spirit and scope of the disclosure. Various embodiments may omit, substitute, or add various procedures or components as appropriate. For instance, the methods described may be performed in an order different from that described, and various steps may be added, omitted, or combined. Also, features described with respect to certain embodiments may be combined in other embodiments.

The detailed description set forth above in connection with the appended drawings describes exemplary embodiments and does not represent the only embodiments that may be implemented or that are within the scope of the claims and present disclosure. The detailed description includes specific details for the purpose of providing an understanding of the described techniques. These techniques, however, may be practiced without these specific details. In some instances, well-known structures and devices are shown in block diagram form in order to avoid obscuring the concepts of the described embodiments.

The various illustrative blocks and modules described in connection with the disclosure herein may be implemented or performed with a general-purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices, e.g., a combination of a DSP and a microprocessor, multiple microprocessors, one or more microprocessors in conjunction with a DSP core, or any other such configuration. A processor may in some cases be in electronic communication with a memory, where the memory stores instructions that are executable by the processor.

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope and spirit of the disclosure and appended claims. For example, due to the nature of software, functions described above can be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items indicates a disjunctive list such that, for example, a list of “at least one of A, B, or C” means A or B or C or AB or AC or BC or ABC (i.e., A and B and C).

A computer program product or computer-readable medium both include a computer-readable storage medium and communication medium, including any mediums that facilitates transfer of a computer program from one place to another. A storage medium may be any medium that can be accessed by a general purpose or special purpose computer. By way of example, and not limitation, computer-readable medium can include RAM, ROM, EEPROM, CD-ROM or other optical disk storage, magnetic disk storage or other magnetic storage devices, or any other medium that can be used to carry or store desired computer-readable program code in the form of instructions or data structures and that can be accessed by a general-purpose or special-purpose computer, or a general-purpose or special-purpose processor. Also, any connection is properly termed a computer-readable medium. For example, if the software is transmitted from a website, server, or other remote light source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium.

The previous description of the disclosure is provided to enable a person skilled in the art to make or use the disclosure. Various modifications to the disclosure will be readily apparent to those skilled in the art, and the generic principles defined herein may be applied to other variations without departing from the spirit or scope of the disclosure. Throughout this disclosure the term “example” or “embodiment” indicates an example or instance and does not imply or require any preference for the noted example. Thus, the disclosure is not to be limited to the examples and designs described herein but is to be accorded the widest scope consistent with the principles and novel features disclosed herein.

What is claimed is:

1. A method for health care monitoring, comprising:
defining a plurality of functions associated with a health care monitoring process;
distributing the plurality of functions associated with the health care monitoring process among a plurality of health care devices; and
communicating among the plurality of health care devices using a network connecting the plurality of health care devices to monitor a patient.

2. The method of claim 1, wherein the distributing the plurality of functions among the plurality of health care devices comprises:
including one or more monitoring modules implementing respective ones of the plurality of defined functions in each of the plurality of health care devices.

3. The method of claim 2, further comprising:
defining a plurality of different topics of data that can be published by each of the respective monitoring modules, wherein the different topics are defined according to a data distribution service protocol.

4. The method of claim 1, wherein the plurality of health care devices cooperatively work together to accomplish all of the plurality of functions associated with the health care monitoring process.

5. The method of claim 1, wherein the network is a wireless network between at least two of the plurality of health care devices.

6. The method of claim 1, wherein the network defines a data bus to which each of the plurality of health care devices may publish certain types data and from which each of the plurality of health care devices may subscribe to certain types of data.

7. The method of claim 6, wherein each health care device that subscribes to a certain type of data is agnostic as to which other health care device published that certain type of data.

8. The method of claim 1, wherein each of the plurality of health care devices is interoperable with a plurality of types of different health care devices.

9. The method of claim 1, wherein the plurality of health care devices includes first and second health care devices, the first and second health care devices each include a first monitoring function, further comprising:
utilizing the first health care device to implement the first function during a first time period; and
switching to utilize the second health care device to implement the first function during a second time period upon an occurrence of a pre-defined event.

10. The method of claim 9, wherein the second health care device is not utilized during the first time period to implement the first function and the first health care device is not utilized during the second time period to implement the first function.

11. The method of claim 9, wherein the pre-defined event relates to loss of power for the first health care device.

12. The method of claim 9, wherein the plurality of health care devices further includes a third health care device including a second monitoring function, and wherein the third health care device is utilized to implement the second monitoring function during both the first and second time periods.

13. The method of claim 9, wherein the switching to utilize the second health care device happens automatically upon the occurrence of the pre-defined event.

14. A health care device, comprising:
a monitoring module configured to perform one of a plurality of defined functions associated with a health care monitoring process; and
a core domain module,
wherein the health care device is configured to communicate with other health care devices over a network, the other health care devices including modules configured to perform others of the plurality of defined functions associated with the health care monitoring process.

15. The device of claim 14, wherein the monitoring module is a data source domain module configured to transmit measurements from a transducer to the communications module for publication on the network.

16. The device of claim 15, wherein the health care device further comprises:
a communications interface configured to communicate with the other health care devices over the network.

17. The device of claim 14, wherein the monitoring module is a user interface module configured to display information received over the network via the communications module.

18. A method for health care monitoring, comprising:
incorporating at least one function of a plurality of defined functions associated with a health care monitoring process into at least one health care device of a plurality of health care devices; and
utilizing the at least one health care device to perform a subset of the plurality of defined functions associated with the health care monitoring process.

19. A distributed health care monitoring system, comprising:
a plurality of health care devices, each of the plurality of health care devices including two or more of a plurality of monitoring modules, each of the plurality of monitoring modules configured to implement a respective function associated with a health care monitoring process; and
a network connecting the plurality of health care devices.

20. The system of claim 19, wherein the network defines a data bus to which each of the plurality of monitoring modules may publish certain types data and from which each of the plurality of monitoring modules may subscribe to certain types data.

21. The system of claim 20, further comprising:
a server coupled with the network and configured to store information published to the data bus by the health care devices.

22. The system of claim 20, wherein one of the plurality of health care devices comprises a sensor device, the sensor device includes a data source domain module and a transducer, and the data source domain module is configured to publish measurements from the transducer onto the data bus.

23. The system of claim 22, wherein one of the plurality of health care devices comprises a display device, the display device includes a user interface domain module and a display, and the user interface domain module is configured to subscribe to the measurements from the data source domain module of the sensor device and further configured to display the measurements on the display.

24. The system of claim 19, wherein none of the plurality of health care devices includes all of the plurality of monitoring modules.

25. The system of claim 19, wherein the plurality of monitoring modules comprises a core domain module, a remote device management domain module, a data source domain module, a parameter domain module, an alarm domain module, a trend domain module, a user interface domain module, a hardware interfaces domain module, and an algorithm module.
